(19) United States
(12) Patent Application Publication
(10) Pub. No.: US 2004/0234968 A1
(43) Pub. Date: Nov. 25, 2004

(54) Plant oil gland nucleic acid molecules and methods of use

(76) Inventors: Rodney B. Croteau, Pullman, WA (US); Bernd Markus Lange, Pullman, WA (US); Mark R. Wildung, Colfax, WA (US)

Correspondence Address: CHRISTENSEN, O'CONNOR, JOHNSON, KINDNESS, PLLC, 1420 FIFTH AVENUE, SUITE 2800, SEATTLE, WA, 98101-2347, US

(21) Appl. No.: 10/468,488

(22) PCT Filed: Jan. 19, 2001
(86) PCT No.: PCT/US01/02567

Related U.S. Application Data
(60) Provisional application No. 60/177,264, filed on Jan. 20, 2000.

(51) Int. Cl. C12Q 1/68; C07H 21/04; C07K 14/415; C12N 5/04

(52) U.S. Cl. 435/6; 435/69.1; 435/320.1; 530/370; 536/23.6; 435/419

(57) ABSTRACT

In one aspect, the present invention provides nucleic acid molecules that each correspond to all or part of a messenger RNA (mRNA) molecule expressed in plant oil gland cells, such as oil gland secretory cells of essential oil plants. In another aspect, the present invention provides nucleic acid molecules that hybridize to one or more of the peppermint oil gland cDNAs disclosed herein (or to the complement of one or more of the peppermint oil gland cDNAs disclosed herein), under stringent conditions. In another aspect, the present invention provides replicable recombinant cloning vehicles comprising a nucleic acid molecule of the present invention. In yet other aspects of the invention, modified host cells are provided that include a recombinant cloning vehicle and/or nucleic acid molecule of the present invention.

### CROSS-REFERENCE TO RELATED APPLICATION

This application claims the benefit of U.S. Provisional Application No. 60/177,264, filed Jan. 20, 2000.

### FIELD OF THE INVENTION

This invention relates to plant oil glands that produce terpenoid essential oils and resins, to proteins expressed in plant oil gland cells and to nucleic acid molecules that encode proteins expressed in plant oil gland cells.

### BACKGROUND OF THE INVENTION

Plant oil glands are highly specialized anatomical structures that are designed for the production and accumulation of terpenoid essential oils and resins (Fahn, New Phytol. 108:229, 1988). While differing somewhat in structural detail from genera to genera, all oil glands contain one or more secretory cells in which the oil or resin is produced, and incorporate an extracellular cavity into which the essential oil or resin is secreted and stored. Id. Although oil glands conduct some aspects of primary metabolism, typical of other plant cells, they express unique genes involved with the structure and regulated development of the glands themselves, with the biosynthesis of essential oils and resins, with the regulation of these specialized processes, and with the intracellular trafficking of these metabolites and their extracellular secretion to the receptacle adapted for storage of these highly lipophilic products.

The large number of terpenoids, including monoterpenes, sesquiterpenes and diterpenes, produced by oil glands have a variety of uses. For example, monoterpenes are utilized as flavoring agents in food products, and as scents in perfumes (Arctander, S., in Perfume and Flavor Materials of Natural Origin, Arctander Publications, Elizabeth, N.J.; Bedoukian, P. Z. in Perfumery and Flavoring Materials, 4th edition, Allured Publications, Wheaton, Ill., 1995; Allured, S., in Flavor and Fragrance Materials, Allured Publications, Wheaton, Ill., 1997). Monoterpenes are also used as intermediates in various industrial processes (Dawson, F. A., in The Amazing Terpenes, Naval Stores Rev., March/April, 6-12, 1994). Monoterpenes are also implicated in the natural defense systems of plants against pests and pathogens (Francke, W. in Muller, P. M. and Lamparsky, D., eds., Perfumes: Art, Science and Technology, Elsevier Applied Science, NY, N.Y., pp. 61-99, 1991; Harborne, J. B., in Harborne, J. B. and Tomas-Barberan, F. A., eds., Ecological Chemistry and Biochemistry of Plant Terpenoids, Clarendon Press, Oxford, pp. 399-426, 1991; Gershenzon, J. and Croteau, R. in Rosenthal, G. A. and Berenbaum, M. R., eds., Herbivores: Their Interactions with Secondary Plant Metabolites, Academic Press, San Diego, pp. 168-220, 1991). There is also substantial evidence that monoterpenes are effective in the prevention and treatment of cancer (Elson, C. E. and S. G. Yu, J. Nutr. 124:607-614, 1994).

Thus, there is a need for compositions and methods that can be used to further investigate, characterize and manipulate the development, physiology and metabolism of plant oil glands. Further, there is a need for nucleic acid sequences that can be used to physically and/or genetically map the locations of genes expressed in plant oil gland cells, especially those genes that are involved with the development and specialized biochemistry of plant oil glands, such as secretory cells. There is also a need for nucleic acid sequences that can be used as probes to isolate full-length, or substantially full-length, cDNA molecules that encode proteins expressed in plant oil gland cells, or that can be used to block the expression of specific messenger RNA molecules expressed in plant oil gland cells, e.g., by antisense suppression.

### SUMMARY OF THE INVENTION

In accordance with the foregoing, cDNA molecules have been synthesized from mRNA isolated from peppermint oil gland cells and sequenced. Thus, in one aspect, the present invention relates to isolated nucleic acid molecules, of at least fifteen nucleotides in length, that correspond to part or all of a messenger RNA (mRNA) molecule expressed in plant oil gland cells, such as oil gland secretory cells of essential oil plants. Representative examples of the nucleic acid molecules of the present invention are set forth in the sequence listing as SEQ ID NOS:1-472. In another aspect, the present invention relates to isolated nucleic acid molecules that include the nucleotide sequence of any one of the nucleic acid molecules set forth in the sequence listing as SEQ ID NOS:1-472. In yet another aspect, the present invention relates to isolated nucleic acid molecules that hybridize under stringent conditions to any one of the nucleic acid molecules set forth in the sequence listing as SEQ ID NOS:1-472, or to the complement of any one of the nucleic acid molecules set forth in the sequence listing as SEQ ID NOS:1-472.

Thus, in one embodiment, the present invention is directed to isolated nucleic acid molecules that hybridize under stringent conditions to any one of the nucleic acid molecules identified herein as SEQ ID NO:29, SEQ ID NO:17, SEQ ID NO:21, SEQ ID NO:66, SEQ ID NO:60, SEQ ID NO:4, SEQ ID NO:7, SEQ ID NO:8, and SEQ ID NO:9, or to the complement of any one of the nucleic acid molecules identified herein as SEQ ID NO:29, SEQ ID NO:17, SEQ ID NO:21, SEQ ID NO:66, SEQ ID NO:60, SEQ ID NO:4, SEQ ID NO:7, SEQ ID NO:8, and SEQ ID NO:9.

In another embodiment, the present invention is directed to isolated nucleic acid molecules that hybridize under stringent conditions to any one of the nucleic acid molecules identified herein as SEQ ID NO:10, SEQ ID NO:11, SEQ ID NO:12, SEQ ID NO:13, SEQ ID NO:14, SEQ ID NO:15, and SEQ ID NO:16, or to the complement of any one of the nucleic acid molecules identified herein as SEQ ID NO:10, SEQ ID NO:11, SEQ ID NO:12, SEQ ID NO:13, SEQ ID NO:14, SEQ ID NO:15, and SEQ ID NO:16.

In yet another embodiment, the present invention is directed to isolated nucleic acid molecules that hybridize under stringent conditions to any one of the nucleic acid molecules identified herein as SEQ ID NO:107, SEQ ID NO:111, SEQ ID NO:102, SEQ ID NO:110, SEQ ID NO:86, SEQ ID NO:76, SEQ ID NO:81, SEQ ID NO:80, SEQ ID NO:95, and SEQ ID NO:97, or to the complement of any one of the nucleic acid molecules identified herein as SEQ ID NO:107, SEQ ID NO:111, SEQ ID NO:102, SEQ ID NO:110, SEQ ID NO:86, SEQ ID NO:76, SEQ ID NO:81, SEQ ID NO:80, SEQ ID NO:95, and SEQ ID NO:97.

A first group of nucleic acid molecules of the present invention includes cDNA molecules that each encode at least part of a protein that may be involved in the deoxyxylulose-5-phosphate pathway which produces isopentenyl diphosphate (IPP) as the central precursor of terpenoid essential oils. Table 1 identifies representative members of the first group of nucleic acid molecules of the present invention.

A second group of nucleic acid molecules of the present invention includes cDNA molecules that each encode at least part of a protein that may be involved in terpene metabolism, including, for example, terpene synthases, oxidoreductases, cytochrome P₄₅₀-dependent oxidoreductases, putative acyltransferases and putative glucosyltransferases which are likely involved in secondary transformation reactions leading to the terpenoid end products of mint essential oils. Table 2 identifies representative members of the second group of nucleic acid molecules of the present invention.

A third group of nucleic acid molecules of the present invention includes DNA sequences that each encode at least part of a transcription factor, or other regulatory protein, that may be involved in the regulation of oil gland development and the control of gene expression in oil gland cells. Table 3 identifies representative members of the third group of nucleic acid molecules of the present invention.

A fourth group of nucleic acid molecules of the present invention includes DNA sequences that each encode at least part of a protein that may be involved in signal transduction and transport processes occurring during the trafficking and secretion of terpenoid essential oils in oil gland cells. Table 4 identifies representative members of the fourth group of nucleic acid molecules of the present invention.

A fifth group of nucleic acid molecules of the present invention includes DNA sequences that encode portions of proteins of diverse, putative function. Table 5 identifies representative members of the fifth group of nucleic acid molecules of the present invention.

In another aspect, the present invention is directed to replicable recombinant cloning vehicles comprising a nucleic acid molecule of the present invention, such as the nucleic acid molecules having the sequences set forth in the sequence listing as SEQ ID NOS:1-472, their complements, or nucleic acid molecules that hybridize (under stringent hybridization conditions) to the nucleic acid molecules having the sequences set forth in the sequence listing as SEQ ID NOS:1-472, or to their complements.

In yet other aspects of the invention, modified host cells are provided that have been transformed, transfected, infected and/or injected with a recombinant cloning vehicle and/or nucleic acid molecule of the present invention. Thus, by way of non-limiting example, the present invention provides for methods of suppressing gene expression by expressing a cDNA molecule of the present invention, in antisense orientation relative to a promoter sequence, in host cells, such as plant oil gland cells. Again by way of non-limiting example, the present invention provides for methods of enhancing expression of plant oil gland cell proteins by expressing one or more cDNA molecules (that encode proteins normally expressed in plant oil gland cells, such as the secretory cells of oil glands of essential oil plants) of the present invention in a host cell, such as a plant oil gland cell.

In another aspect, the present invention is directed to isolated proteins (such as isolated proteins encoded by cDNA molecules of the present invention) that are naturally expressed in plant oil gland cells.

The inventive concepts described herein may be used, for example, to physically and/or genetically map a plant genome (such as the peppermint plant genome), to isolate full-length (or substantially full-length) cDNA molecules encoding proteins expressed in plant oil gland cells, to isolate genes encoding proteins expressed in plant oil gland cells, to suppress the expression of mRNA molecules expressed in plant oil gland cells (for example by antisense suppression), to enhance expression of plant oil gland cell proteins (for example by genetically transforming a plant cell with a replicable expression vector of the present invention that expresses one or more proteins that are naturally expressed in plant oil gland cells), to enhance or suppress terpenoid essential oil and/or resin production in plant oil glands, to express plant oil gland proteins in bacterial and/or yeast cells to produce plant oil gland products (such as terpenoid essential oils and resins), or to otherwise alter the development, physiology and/or biochemistry of plant cells, such as the oil gland cells of essential oil plants.

### DETAILED DESCRIPTION OF THE PREFERRED EMBODIMENT

As used herein, the terms “amino acid” and “amino acids” refer to all naturally occurring L-α-amino acids or their residues. The amino acids are identified by either the single-letter or three-letter designations:

| | | | | | |
|---|---|---|---|---|---|
| Asp | D | aspartic acid | Ile | I | isoleucine |
| Thr | T | threonine | Leu | L | leucine |
| Ser | S | serine | Tyr | Y | tyrosine |
| Glu | E | glutamic acid | Phe | F | phenylalanine |
| Pro | P | proline | His | H | histidine |
| Gly | G | glycine | Lys | K | lysine |
| Ala | A | alanine | Arg | R | arginine |
| Cys | C | cysteine | Trp | W | tryptophan |
| Val | V | valine | Gln | Q | glutamine |
| Met | M | methionine | Asn | N | asparagine |

As used herein, the term “nucleotide” means a monomeric unit of DNA or RNA containing a sugar moiety (pentose), a phosphate and a nitrogenous heterocyclic base. The base is linked to the sugar moiety via the glycosidic carbon (1′ carbon of pentose) and that combination of base and sugar is called a nucleoside. The base characterizes the nucleotide with the four bases of DNA being adenine (“A”), guanine (“G”), cytosine (“C”) and thymine (“T”). Inosine (“I”) is a synthetic base that can be used to substitute for any of the four, naturally-occurring bases (A, C, G or T). The four RNA bases are A, G, C and uracil (“U”). The nucleotide sequences described herein comprise a linear array of nucleotides connected by phosphodiester bonds between the 3′ and 5′ carbons of adjacent pentoses. The one letter codes for nucleotide sequences used herein are set forth at page 300 of the present application.

“Oligonucleotide” refers to short length single or double stranded sequences of deoxyribonucleotides linked via phosphodiester bonds. The oligonucleotides are chemically synthesized by known methods and purified, for example, on polyacrylamide gels.

The term “hybridize under stringent conditions”, and grammatical equivalents thereof, means that a nucleic acid molecule that has hybridized to a target nucleic acid molecule immobilized on a DNA or RNA blot (such as a Southern blot or Northern blot) remains hybridized to the immobilized target molecule on the blot during washing of the blot under stringent conditions. In this context, exemplary hybridization conditions are: hybridization at 65° C. in 5.0×SSC, 1% sodium dodecyl sulfate, for 16 hours (lower stringency hybridizations preferably utilize 6.0×SSC, 1% sodium dodecyl sulfate, at 20° C. to 30° C. for 16 hours). Exemplary very high stringency conditions for washing DNA or RNA blots are: two washes of fifteen minutes each at 20° C. to 30° C. in 2.0×SSC, followed by two washes of twenty minutes each at 65° C. in 0.5×SSC. Exemplary high stringency conditions for washing DNA or RNA blots are: two washes of twenty minutes each at 20° C. to 30° C. in 2.0×SSC, followed by one wash of thirty minutes at 55° C. in 1.0×SSC. Exemplary moderate stringency conditions for washing DNA or RNA blots are: two washes of twenty minutes each at 20° C. to 30° C. in 3.0×SSC. Preferably, moderate stringency wash conditions are utilized after hybridization in lower stringency hybridization conditions, i.e., 6.0×SSC, 1% sodium dodecyl sulfate, at 20° C. to 30° C. for 16 hours.

The term “essential oil plant,” or “essential oil plants,” refers to a group of plant species that produce high levels of monoterpenoid and/or sesquiterpenoid and/or diterpenoid oils, and/or high levels of monoterpenoid and/or sesquiterpenoid and/or diterpenoid resins. The foregoing oils and/or resins account for greater than about 0.005% of the fresh weight of an essential oil plant that produces them. The essential oils and/or resins are more fully described, for example, in E. Guenther, The Essential Oils, Vols. I-VI, R. E. Krieger Publishing Co., Huntington N.Y., 1975, incorporated herein by reference. The essential oil plants include, but are not limited to:

Lamiaceae, including, but not limited to, the following species: Ocimum (basil), Lavandula (Lavender), Origanum (oregano), Mentha (mint), Salvia (sage), Rosmarinus, (rosemary), Thymus (thyme), Satureja (savory), Monarda (balm) and Melissa.

Umbelliferae, including, but not limited to, the following species: Carum (caraway), Anethum (dill), foeniculum (fennel) and Daucus (carrot).

Asteraceae (Compositae), including, but not limited to, the following species: Artemisia (tarragon, sage brush), Tanacetum (tansy).

Rutaceae (e.g., Citrus plants); Rosaceae (e.g., roses); Myrtaceae (e.g., Eucalyptus, Melaleuca); the Gramineae (e.g., Cymbopogon (citronella)); Geranaceae (Geranium) and certain conifers including Abies (e.g., Canadian balsam), Cedrus (cedar), Thuja, Juniperus, Pinus (pines) and Picea (spruces).

The range of essential oil plants is more fully set forth in E. Guenther, The Essential Oils, Vols. I-VI, R. E. Krieger Publishing Co., Huntington N.Y., 1975, which is incorporated herein by reference.

The term “angiosperm” refers to a class of plants that produce seeds that are enclosed in an ovary.

The term “gymnosperm” refers to a class of plants that produce seeds that are not enclosed in an ovary.

The abbreviation “SSC” refers to a buffer used in nucleic acid hybridization solutions. One liter of the 20×(twenty times concentrate) stock SSC buffer solution (pH 7.0) contains 175.3 g sodium chloride and 88.2 g sodium citrate.

The terms “alteration”, “amino acid sequence alteration”, “variant” and “amino acid sequence variant” refer to protein molecules with some differences in their amino acid sequences as compared to the corresponding, native, i.e., naturally-occurring, proteins. Ordinarily, the variants will possess at least about 70% identity with the corresponding native proteins, and preferably, they will be at least about 80% identical to the corresponding, native proteins. The amino acid sequence variants falling within this invention possess substitutions, deletions, and/or insertions at certain positions. Sequence variants may be used to attain desired enhanced or reduced enzymatic activity, modified regiochemistry or stereochemistry, or altered substrate utilization or product distribution.

Substitutional protein variants are those that have at least one amino acid residue in the native protein sequence removed and a different amino acid inserted in its place at the same position. The substitutions may be single, where only one amino acid in the molecule has been substituted, or they may be multiple, where two or more amino acids have been substituted in the same molecule. Substantial changes in the activity of the proteins of the present invention may be obtained by substituting an amino acid with a side chain that is significantly different in charge and/or structure from that of the native amino acid. This type of substitution would be expected to affect the structure of the polypeptide backbone and/or the charge or hydrophobicity of the molecule in the area of the substitution.

Moderate changes in the activity of the proteins of the present invention would be expected by substituting an amino acid with a side chain that is similar in charge and/or structure to that of the native molecule. This type of substitution, referred to as a conservative substitution, would not be expected to substantially alter either the structure of the polypeptide backbone or the charge or hydrophobicity of the molecule in the area of the substitution.

Insertional protein variants are those with one or more amino acids inserted immediately adjacent to an amino acid at a particular position in the native protein. Immediately adjacent to an amino acid means connected to either the α-carboxy or α-amino functional group of the amino acid. The insertion may be one or more amino acids. Ordinarily, the insertion will consist of one or two conservative amino acids. Amino acids similar in charge and/or structure to the amino acids adjacent to the site of insertion are defined as conservative. Alternatively, this invention includes insertion of an amino acid with a charge and/or structure that is substantially different from the amino acids adjacent to the site of insertion.

Deletional variants are those where one or more amino acids in the native proteins have been removed. Ordinarily, deletional variants will have one or two amino acids deleted in a particular region of the protein.

The terms “DNA sequence encoding”, “DNA encoding” “nucleic acid molecule encoding” and “nucleic acid encoding” refer to the order or sequence of deoxyribonucleotides along a strand of deoxyribonucleic acid. The order of these deoxyribonucleotides determines the order of amino acids along the translated polypeptide chain. The DNA sequence thus codes for the amino acid sequence.

The terms “replicable vector” “replicable expression vector” and “expression vector” refer to a piece of DNA, usually double-stranded, which may have inserted into it another piece of DNA (the insert DNA) such as, but not limited to, a cDNA molecule. The vector is used to transport the insert DNA into a suitable host cell. The insert DNA may be derived from the host cell, or may be derived from a different cell or organism. Once in the host cell, the vector can replicate independently of or coincidental with the host chromosomal DNA, and several copies of the vector and its inserted DNA may be generated. The terms “replicable expression vector” and “expression vector” refer to vectors that contain the necessary elements that permit transcribing and translating the insert DNA into a polypeptide. Many molecules of the polypeptide encoded by the insert DNA can thus be rapidly synthesized.

The terms “transformed host cell,” “transformed” and “transformation” refer to the introduction of DNA into a cell. The cell is termed a “host cell”, and it may be, for example, a prokaryotic or a eukaryotic cell. Typical prokaryotic host cells include various strains of E. coli. Typical eukaryotic host cells are plant cells, such as maize cells, yeast cells, insect cells or animal cells. The introduced DNA is usually in the form of a vector containing an inserted piece of DNA. The introduced DNA sequence may be from the same species as the host cell or from a different species from the host cell, or it may be a hybrid DNA sequence, containing some foreign DNA and some DNA derived from the host species.

In one aspect, the present invention relates to isolated nucleic acid molecules (such as cDNA molecules and genomic clones) that each correspond to all or part of a messenger RNA (mRNA) molecule expressed in a plant oil gland cell, such as oil gland secretory cells. Representative examples of the nucleic acid molecules of the present invention are set forth in SEQ ID NOS:1-472 which disclose full and partial length cDNA molecules synthesized from mRNA extracted from peppermint oil gland cells. Full length cDNAs of the present invention may be obtained, for example, by utilizing the technique of RACE (Rapid Amplification of cDNA Ends), also known as Anchored-PCR. For example, the missing 5′-end of a partial-length cDNA molecule of the present invention can be obtained by priming first strand DNA synthesis with an mRNA-specific oligonucleotide based on the sequence of a portion of the cloned, partial-length cDNA. A poly(A) tail is appended to the 3′-end of the first strand cDNA using terminal deoxynucleotidyltransferase, and second strand cDNA synthesis is primed using a second strand primer that includes a 3′ oligo(dT) portion and a unique oligonucleotide sequence (a representative example of such a “hybrid” primer has the following nucleotide sequence: 5′-CCAGTGAGCAGAGTGACGAGGACTCGAGCTCAAGCTTTTTTTTTTTTTTTTT-3′) (SEQ ID NO:473). Subsequent amplifications can be primed using the unique portion of the second strand primer and a gene-specific primer upstream of and distinct from the primer used for first strand cDNA synthesis, i.e., the upstream gene-specific primer is closer to the 5′-end of the target cDNA molecule than the primer used for first strand cDNA synthesis). A representative RACE protocol is set forth in Chapter 2 of The Polymerase Chain Reaction (Mullis et al., eds.), Birkhauser Boston (1994), which chapter is incorporated herein by reference.

Full length cDNAs of the present invention may also be cloned, for example, by utilizing the technique of hybridizing radiolabelled nucleic acid probes to nucleic acids immobilized on nitrocellulose filters or nylon membranes, as set forth, for example, at pages 9.52 to 9.55 of Molecular Cloning, A Laboratory Manual (2nd edition), J. Sambrook et al. eds., the cited pages of which are incorporated herein by reference. A representative protocol (based on the aforementioned Sambrook et al. publication) for hybridizing radiolabelled nucleic acid probes to nucleic acids immobilized on nitrocellulose filters or nylon membranes is set forth in Example 2 herein. For example, a full-length cDNA, or substantially full-length cDNA that includes all of the coding region, homologous to one of the cDNAs set forth in SEQ ID NOS:1-472 can be cloned by screening a peppermint oil gland cell cDNA library with the appropriate cDNA from the cDNA sequences set forth in SEQ ID NOS:1-472 using the foregoing hybridization technique. Exemplary hybridization and wash conditions useful for screening the oil gland cDNA library are as follows. Hybridization at 65° C. in 5.0×SSC, 1% sodium dodecyl sulfate, for 16 hours (lower stringency hybridizations preferably utilize 6.0×SSC, 1% sodium dodecyl sulfate, at 20° C. to 30° C. for 16 hours). Exemplary very high stringency wash conditions for screening the oil gland cDNA library are: two washes of fifteen minutes each at 20° C. to 30° C. in 2.0×SSC, followed by two washes of twenty minutes each at 65° C. in 0.5×SSC. Exemplary high stringency wash conditions for screening the oil gland cDNA library are: two washes of twenty minutes each at 20° C. to 30° C. in 2.0×SSC, followed by one wash of thirty minutes at 55° C. in 1.0×SSC. Exemplary moderate stringency wash conditions for screening the oil gland cDNA library are: two washes of twenty minutes each at 20° C. to 30° C. in 3.0×SSC. Preferably, moderate stringency wash conditions are utilized after hybridization in lower stringency hybridization conditions, i.e., 6.0×SSC, 1% sodium dodecyl sulfate, at 20° C. to 30° C. for 16 hours.

Full length genes of the present invention may be cloned, for example, by utilizing partial-length nucleotide sequences of the invention and various methods known in the art. Gobinda et al. (PCR Methods Applic. 2:318-22, 1993), incorporated herein by reference, disclose “restriction-site PCR” as a direct method which uses universal primers to retrieve unknown sequence adjacent to a known locus. First, genomic DNA is amplified in the presence of a linker-primer, that is homologous to a linker sequence ligated to the ends of the genomic DNA fragments, and in the presence of a primer specific to the known region. The amplified sequences are subjected to a second round of PCR with the same linker primer and another specific primer internal to the first one. Products of each round of PCR are transcribed with an appropriate RNA polymerase and sequenced using reverse transcriptase.

Inverse PCR permits acquisition of unknown sequences starting with primers based on a known region (Triglia, T. et al., Nucleic Acids Res. 16:8186, 1988, incorporated herein by reference). The method uses several restriction enzymes to generate a suitable fragment in the known region of a gene. The fragment is then circularized by intramolecular ligation and used as a PCR template. Divergent primers are designed from the known region.

Capture PCR (Lagerstrom, M. et al., PCR Methods Applic. 1:111-19, 1991, incorporated herein by reference) is a method for PCR amplification of DNA fragments adjacent to a known sequence in human and YAC DNA. Capture PCR also requires multiple restriction enzyme digestions and ligations to place an engineered double-stranded sequence into an unknown portion of the DNA molecule before PCR.

The present invention also relates to nucleic acid molecules that hybridize under stringent conditions to one or more of the nucleic acid molecules (or to their complements) that are set forth in SEQ ID NOS:1-472 of the present application. A representative hybridization protocol utilizes the technique of hybridizing radiolabelled nucleic acid probes to nucleic acids immobilized on nitrocellulose filters or nylon membranes as set forth at pages 9.52 to 9.55 of Molecular Cloning, A Laboratory Manual (2nd edition), J. Sambrook et al. eds., the cited pages of which are incorporated herein by reference. Example 2 herein sets forth a representative protocol useful for identifying nucleic acid molecules that hybridize under stringent conditions to one or more of the nucleic acid molecules (or to their complements) that are set forth in SEQ ID NOS:1-472 of the present application. Representative hybridization probes include fragments, of at least 15 nucleotides in length, of the DNA molecules (or their antisense complements) having the sequences set forth in SEQ ID NOS:1-472. Thus, for example, the DNA molecules having the sequences set forth in SEQ ID NOS:1-472 can be used as hybridization probes.

Such hybridization probes may be labelled with appropriate reporter molecules. Means for producing specific hybridization probes include oligolabelling, nick translation, end-labelling or PCR amplification using a labelled nucleotide.

Exemplary hybridization and wash conditions useful for identifying (by Southern blotting) nucleic acid molecules of the invention that hybridize to one or more of the nucleic acid molecules (or to their complements) that are set forth in SEQ ID NOS:1-472 are as follows. Hybridization at 65° C. in 5.0×SSC, 1% sodium dodecyl sulfate, for 16 hours (lower stringency hybridizations preferably utilize 6.0×SSC, 1% sodium dodecyl sulfate, at 20° C. to 30° C. for 16 hours). Exemplary very high stringency wash conditions are: two washes of fifteen minutes each at 20° C. to 30° C. in 2.0×SSC, followed by two washes of twenty minutes each at 65° C. in 0.5×SSC. Exemplary high stringency wash conditions are: two washes of twenty minutes each at 20° C. to 30° C. in 2.0×SSC, followed by one wash of thirty minutes at 55° C. in 1.0×SSC. Exemplary moderate stringency wash conditions are: two washes of twenty minutes each at 20° C. to 30° C. in 3.0×SSC. Preferably, moderate stringency wash conditions are utilized after hybridization in lower stringency hybridization conditions, i.e., 6.0×SSC, 1% sodium dodecyl sulfate, at 20° C. to 30° C. for 16 hours.

Nucleic acid molecules of the present invention can be isolated by using a variety of cloning techniques known to those of ordinary skill in the art. Thus, for example, nucleic acid molecules of the present invention can be isolated by using the DNA molecules, having the sequences set forth in SEQ ID NOS:1-472, as hybridization probes to screen cDNA or genomic libraries utilizing the aforementioned technique of hybridizing radiolabelled nucleic acid probes to nucleic acids immobilized on nitrocellulose filters or nylon membranes. Exemplary hybridization and wash conditions are: hybridization at 65° C. in 3.0×SSC, 1% sodium dodecyl sulfate; washing (three washes of twenty minutes each at 55° C.) in 0.5×SSC, 1% (w/v) sodium dodecyl sulfate.

Again, by way of example, nucleic acid molecules of the present invention can be isolated by the polymerase chain reaction (PCR) described in The Polymerase Chain Reaction (Mullis et al. eds.), Birkhauser Boston (1994), incorporated herein by reference. Thus, for example, first strand DNA synthesis can be primed using an oligo(dT) primer, and second strand cDNA synthesis can be primed using an oligonucleotide primer that corresponds to a portion of the 5′-untranslated region of a cDNA molecule that is homologous to the target DNA molecule. Subsequent rounds of PCR can be primed using the second strand cDNA synthesis primer and a primer that corresponds to a portion of the 3′-untranslated region of a cDNA molecule that is homologous to the target DNA molecule. In this way, homologs of a cDNA molecule can be cloned from a range of different plant species.

By way of non-limiting example, representative PCR reaction conditions for amplifying nucleic acid molecules of the present invention (such as amplifying genes from plant genomic DNA) are as follows. The following reagents are mixed in a tube (on ice) to form the PCR reaction mixture: DNA template (e.g., up to 1 μg genomic DNA, or up to 0.1 μg cDNA), 0.1-0.3 mM dNTPs, 10 μl 10×PCR buffer (10×PCR buffer contains 500 mM KCL, 15 mM MgCL₂, 100 mM Tris-HCL, pH 8.3), 50 pmol of each PCR primer (PCR primers should preferably be greater than 20 bp in length and have a degeneracy of 102 to 103), 2.5 units of Taq DNA polymerase (Perkin Elmer, Norwalk, Conn.) and deionized water to a final volume of 50 μl. The tube containing the reaction mixture is placed in a thermocycler and a thermocycler program is run as follows. Denaturation at 94° C. for 2 minutes, then 30 cycles of: 94° C. for 30 seconds, 47° C. to 55° C. for 30 seconds, and 72° C. for 30 seconds to two and a half minutes.

Further, nucleic acid molecules of the present invention can also be isolated, for example, by utilizing antibodies that recognize the protein encoded by the nucleic acid molecule. By way of non-limiting example, a cDNA expression library can be screened using antibodies in order to identify one or more clones that encode a protein recognized by the antibodies. DNA expression library technology is well known to those of ordinary skill in the art. An exemplary protocol for screening a cDNA expression library is set forth in Example 3 herein. Screening cDNA expression libraries is fully discussed in Chapter 12 of Sambrook et al. (1989) Molecular Cloning: A Laboratory Manual, 2nd ed., Cold Spring Harbor Laboratory, Cold Spring Harbor, N.Y., the cited chapter of which is incorporated herein by reference.

By way of representative example, antigen useful for raising antibodies for screening expression libraries can be prepared in the following manner. A full-length cDNA molecule of the present invention (or a cDNA molecule of the invention that is not full-length, but which includes all of the coding region) can be cloned into a plasmid vector, such as a Bluescript plasmid (available from Stratagene, Inc., La Jolla, Calif.). The recombinant vector is then introduced into an E. coli strain (such as E. coli XL1-Blue, also available from Stratagene, Inc.) and the protein encoded by the cDNA is expressed in E. coli and then purified. For example, E. coli XL 1-Blue harboring a Bluescript vector including a cDNA molecule of interest can be grown overnight at 37° C. in LB medium containing 100 μg ampicillin/ml. A 50 μl aliquot of the overnight culture can be used to inoculate 5 ml of fresh LB medium containing ampicillin, and the culture grown at 37° C. with vigorous agitation to A₆₀₀=0.5 before induction with 1 mM IPTG. After an additional two hours of growth, the suspension is centrifuged (1000×g, 15 min, 4° C.), the media removed, and the pelleted cells resuspended in 1 ml of cold buffer that preferably contains 1 mM EDTA and one or more proteinase inhibitors, such as those described herein in connection with the purification of the isolated proteins of the present invention. The cells can be disrupted by sonication with a microprobe. The chilled sonicate is cleared by centrifugation and the expressed, recombinant protein purified from the supernatant by art-recognized protein purification techniques, such as those described herein.

Methods for preparing monoclonal and polyclonal antibodies are well known to those of ordinary skill in the art and are set forth, for example, in chapters five and six of Antibodies A Laboratory Manual, E. Harlow and D. Lane, Cold Spring Harbor Laboratory (1988), the cited chapters of which are incorporated herein by reference. In one representative example, polyclonal antibodies specific for a purified protein can be raised in a New Zealand rabbit implanted with a whiffle ball. One μg of protein is injected at intervals directly into the whiffle ball granuloma. A representative injection regime is injections (each of 1 μg protein) at day 1, day 14 and day 35. Granuloma fluid is withdrawn one week prior to the first injection (preimmune serum), and forty days after the final injection (postimmune serum).

Nucleic acid molecules of the present invention can be used for a variety of purposes including, but not limited to: isolation of full-length cDNAs (and/or complete genes) encoding proteins expressed in plant oil gland cells, such as the oil gland secretory cells of essential oil plants; the development of efficient expression systems for proteins normally expressed in plant oil gland cells; investigation and/or manipulation of the developmental regulation of proteins normally expressed in plant oil gland cells; to express plant oil gland proteins in bacterial and/or yeast cells to produce plant oil gland products (such as terpenoid essential oils and resins); genetic transformation of a wide range of organisms, including plants, and to physically and/or genetically map a plant genome (such as the peppermint plant genome). A nucleic acid molecule of the present invention may be incorporated into plants, or cell cultures derived therefrom, for a variety of purposes including enhancement or suppression (for example by antisense suppression) of expression of proteins normally expressed in plant oil glands and which are involved in the biosynthesis of terpenoid essential oils and resins. Thus, for example, in one aspect the present invention provides methods for enhancing the production of essential oils and/or resins in plants by overexpressing a protein involved in the biosynthesis of terpenoid essential oils and/or resins in plant oil gland cells. By way of non-limiting example, nucleic acid molecules of the present invention that encode proteins involved in lipid secretion (i.e., extracellular transport), or proteins involved in intracellular transport of lipids, or transcription factors that regulate terpenoid biosynthesis, may be introduced into cultured cells (such as cells cultured in liquid medium) of the plant species Taxus which synthesize the diterpene paclitaxel (or may be introduced into microorganisms such as Taxomyces andreanae and Penicillium raistrickii which synthesize the diterpene paclitaxel) thereby enhancing the amount of paclitaxel produced and/or secreted by the cultured cells. Representative examples of nucleic acid molecules of the present invention that encode putative transcription factors are set forth in Table 3 herein. Representative examples of nucleic acid molecules of the present invention that encode proteins believed to be involved in lipid secretion (i.e., extracellular lipid transport), or proteins believed to be involved in intracellular transport of lipids are set forth in Table 4 herein.

In another aspect, the present invention is directed to isolated proteins (such as proteins encoded by the nucleic acid molecules of the present invention) that are naturally expressed in plant oil gland cells. The proteins of the present invention can be isolated, for example, by incorporating a nucleic acid molecule of the invention (such as a cDNA molecule) into an expression vector, introducing the expression vector into a host cell and expressing the nucleic acid molecule to yield protein. The protein can then be purified by art-recognized means. When a crude protein extract is initially prepared, it may be desirable to include one or more proteinase inhibitors in the extract. Representative examples of proteinase inhibitors include: serine proteinase inhibitors (such as phenylmethylsulfonyl fluoride (PMSF), benzamide, benzamidine HCl, ε-Amino-n-caproic acid and aprotinin (Trasylol)); cysteine proteinase inhibitors, such as sodium p-hydroxymercuribenzoate; competitive proteinase inhibitors, such as antipain and leupeptin; covalent proteinase inhibitors, such as iodoacetate and N-ethylmaleimide; aspartate (acidic) proteinase inhibitors, such as pepstatin and diazoacetylnorleucine methyl ester (DAN); metalloproteinase inhibitors, such as EGTA [ethylene glycol bis(β-aminoethyl ether) N,N,N′,N′-tetraacetic acid], and the chelator 1,10-phenanthroline.

Representative examples of art-recognized techniques for purifying, or partially purifying, proteins from biological material are exclusion chromatography, ion-exchange chromatography, hydrophobic interaction chromatography, reversed-phase chromatography and immobilized metal affinity chromatography.

Hydrophobic interaction chromatography and reversed-phase chromatography are two separation methods based on the interactions between the hydrophobic moieties of a sample and an insoluble, immobilized hydrophobic group present on the chromatography matrix. In hydrophobic interaction chromatography the matrix is hydrophilic and is substituted with short-chain phenyl or octyl nonpolar groups. The mobile phase is usually an aqueous salt solution. In reversed phase chromatography the matrix is silica that has been substituted with longer n-alkyl chains, usually C₈ (octylsilyl) or C₁₈ (octadecylsilyl). The matrix is less polar than the mobile phase. The mobile phase is usually a mixture of water and a less polar organic modifier.

Separations on hydrophobic interaction chromatography matrices are usually done in aqueous salt solutions, which generally are nondenaturing conditions. Samples are loaded onto the matrix in a high-salt buffer and elution is by a descending salt gradient. Separations on reversed-phase media are usually done in mixtures of aqueous and organic solvents, which are often denaturing conditions. In the case of protein and/or peptide purification, hydrophobic interaction chromatography depends on surface hydrophobic groups and is carried out under conditions which maintain the integrity of the protein molecule. Reversed-phase chromatography depends on the native hydrophobicity of the protein and is carried out under conditions which expose nearly all hydrophobic groups to the matrix, i.e., denaturing conditions.

Ion-exchange chromatography is designed specifically for the separation of ionic or ionizable compounds. The stationary phase (column matrix material) carries ionizable functional groups, fixed by chemical bonding to the stationary phase. These fixed charges carry a counterion of opposite sign. This counterion is not fixed and can be displaced. Ion-exchange chromatography is named on the basis of the sign of the displaceable charges. Thus, in anion ion-exchange chromatography the fixed charges are positive and in cation ion-exchange chromatography the fixed charges are negative.

Retention of a molecule on an ion-exchange chromatography column involves an electrostatic interaction between the fixed charges and those of the molecule, binding involves replacement of the nonfixed ions by the molecule. Elution, in turn, involves displacement of the molecule from the fixed charges by a new counterion with a greater affinity for the fixed charges than the molecule, and which then becomes the new, nonfixed ion.

The ability of counterions (salts) to displace molecules bound to fixed charges is a function of the difference in affinities between the fixed charges and the nonfixed charges of both the molecule and the salt. Affinities in turn are affected by several variables, including the magnitude of the net charge of the molecule and the concentration and type of salt used for displacement.

Solid-phase packings used in ion-exchange chromatography include cellulose, dextrans, agarose, and polystyrene. The exchange groups used include DEAE (diethylaminoethyl), a weak base, that will have a net positive charge when ionized and will therefore bind and exchange anions; and CM (carboxymethyl), a weak acid, with a negative charge when ionized that will bind and exchange cations. Another form of weak anion exchanger contains the PEI (polyethyleneimine) functional group. This material, most usually found on thin layer sheets, is useful for binding proteins at pH values above their pI. The polystyrene matrix can be obtained with quaternary ammonium functional groups for strong base anion exchange or with sulfonic acid functional groups for strong acid cation exchange. Intermediate and weak ion-exchange materials are also available. Ion-exchange chromatography need not be performed using a column, and can be performed as batch ion-exchange chromatography with the slurry of the stationary phase in a vessel such as a beaker.

Gel filtration is performed using porous beads as the chromatographic support. A column constructed from such beads will have two measurable liquid volumes, the external volume, consisting of the liquid between the beads, and the internal volume, consisting of the liquid within the pores of the beads. Large molecules will equilibrate only with the external volume while small molecules will equilibrate with both the external and internal volumes. A mixture of molecules (such as proteins) is applied in a discrete volume or zone at the top of a gel filtration column and allowed to percolate through the column. The large molecules are excluded from the internal volume and therefore emerge first from the column while the smaller molecules, which can access the internal volume, emerge later. The volume of a conventional matrix used for protein purification is typically 30 to 100 times the volume of the sample to be fractionated. The absorbance of the column effluent can be continuously monitored at a desired wavelength using a flow monitor.

A technique that is often applied to the purification of proteins is High Performance Liquid Chromatography (HPLC). HPLC is an advancement in both the operational theory and fabrication of traditional chromatographic systems. HPLC systems for the separation of biological macromolecules vary from the traditional column chromatographic systems in three ways; (1) the column packing materials are of much greater mechanical strength, (2) the particle size of the column packing materials has been decreased 5- to 10-fold to enhance adsorption-desorption kinetics and diminish bandspreading, and (3) the columns are operated at 10-60 times higher mobile-phase velocity. Thus, by way of non-limiting example, HPLC can utilize exclusion chromatography, ion-exchange chromatography, hydrophobic interaction chromatography, reversed-phase chromatography and immobilized metal affinity chromatography. Art-recognized techniques for the purification of proteins and peptides are set forth in Methods in Enzymology, Vol. 182, Guide to Protein Purification, Murray P. Deutscher, ed. (1990), which publication is incorporated herein by reference. In particular, Section IV, chapter 14, of the Deutscher publication discloses representative techniques for the preparation of protein extracts from plant material.

In addition to native proteins, protein variants produced by deletions, substitutions, mutations and/or insertions are intended to be within the scope of the invention except insofar as limited by the prior art. In the design of a particular site directed mutagenesis experiment, it is generally desirable to first make a non-conservative substitution (e.g., Ala for Cys, His or Glu) and determine if the biological activity of the mutated protein is greatly impaired as a consequence. The properties of the mutagenized protein are then examined with particular attention to the kinetic parameters of Kₘ and k_{cat} as sensitive indicators of altered function, from which changes in binding and/or catalysis per se may be deduced by comparison to the native enzyme. If the residue is by this means demonstrated to be important by activity impairment, or knockout, then conservative substitutions can be made, such as Asp for Glu to alter side chain length, Ser for Cys, or Arg for His. For hydrophobic segments, it is largely size that is usefully altered, although aromatics can also be substituted for alkyl side chains. Changes in the normal product distribution can indicate which step(s) of the reaction sequence have been altered by the mutation. Modification of the hydrophobic pocket can be employed to change binding conformations for substrates and result in altered regiochemistry and/or stereochemistry.

The protein variants of this invention may be constructed by mutating the DNA sequences that encode the wild-type proteins, such as by using techniques commonly referred to as site-directed mutagenesis. Nucleic acid molecules encoding the proteins of the present invention can be mutated by a variety of PCR techniques well known to one of ordinary skill in the art. (See, for example, the following publications, the cited portions of which are incorporated by reference herein: PCR Strategies, M. A. Innis et al. eds., 1995, Academic Press, San Diego, Calif. (Chapter 14); PCR Protocols: A Guide to Methods and Applications, M. A. Innis et al. eds., Academic Press, NY (1990).)

By way of non-limiting example, the two primer system utilized in the Transformer Site-Directed Mutagenesis kit from Clontech (Palo Alto, Calif.), may be employed for introducing site-directed mutants into nucleic acid molecules that encode proteins of the present invention. Following denaturation of the target plasmid in this system, two primers are simultaneously annealed to the plasmid; one of these primers contains the desired site-directed mutation, the other contains a mutation at another point in the plasmid resulting in elimination of a restriction site. Second strand synthesis is then carried out, tightly linking these two mutations, and the resulting plasmids are transformed into a mutS strain of E. coli. Plasmid DNA is isolated from the transformed bacteria, restricted with the relevant restriction enzyme (thereby linearizing the unmutated plasmids), and then retransformed into E. coli. This system allows for generation of mutations directly in an expression plasmid, without the necessity of subcloning or generation of single-stranded phagemids. The tight linkage of the two mutations and the subsequent linearization of unmutated plasmids results in high mutation efficiency and allows minimal screening. Following synthesis of the initial restriction site primer, this method requires the use of only one new primer type per mutation site. Rather than prepare each positional mutant separately, a set of “designed degenerate” oligonucleotide primers can be synthesized in order to introduce all of the desired mutations at a given site simultaneously. Transformants can be screened by sequencing the plasmid DNA through the mutagenized region to identify and sort mutant clones. Each mutant DNA can then be fully sequenced or restricted and analyzed by electrophoresis on Mutation Detection Enhancement gel (J. T. Baker, Sanford, Me.) to confirm that no other alterations in the sequence have occurred (by band shift comparison to the unmutagenized control).

Again, by way of non-limiting example, the two primer system utilized in the QuikChange™ Site-Directed Mutagenesis kit from Stratagene (LaJolla, Calif.), may be employed for introducing site-directed mutations into nucleic acid molecules that encode proteins of the present invention. Double-stranded plasmid DNA, containing the insert bearing the target mutation site, is denatured and mixed with two oligonucleotides complementary to each of the strands of the plasmid DNA at the target mutation site. The annealed oligonucleotide primers are extended using Pfu DNA polymerase, thereby generating a mutated plasmid containing staggered nicks. After temperature cycling, the unmutated, parental DNA template is digested with restriction enzyme DpnI which cleaves methylated or hemimethylated DNA, but which does not cleave unmethylated DNA. The parental, template DNA is almost always methylated or hemimethylated since most strains of E. coli, from which the template DNA is obtained, contain the required methylase activity. The remaining, annealed vector DNA incorporating the desired mutation(s) is transformed into E. coli.

Nucleic acid molecules encoding proteins of the present invention (including variants of the naturally-occurring proteins) can be cloned into a pET (or other) overexpression vector that can be employed to transform E. coli, such as E. coli strain BL21(DE3)pLysS, for high level production of the protein, and purification by standard protocols. Examples of plasmid vectors and E. coli strains that can be used to express high levels of the proteins of the present invention are set forth in Sambrook et al., Molecular Cloning, A Laboratory Manual, 2nd Edition (1989), Chapter 17. The method of FAB-MS mapping can be employed to rapidly check the fidelity of protein expression. This technique provides for sequencing segments throughout the whole protein and provides the necessary confidence in the sequence assignment. In a mapping experiment of this type, protein is digested with a protease (the choice will depend on the specific region to be modified since this segment is of prime interest and the remaining map should be identical to the map of unmutagenized protein). The set of cleavage fragments is fractionated by microbore HPLC (reversed phase or ion exchange, again depending on the specific region to be modified) to provide several peptides in each fraction, and the molecular weights of the peptides are determined by FAB-MS. The masses are then compared to the molecular weights of peptides expected from the digestion of the predicted sequence, and the correctness of the sequence quickly ascertained. Since the exemplary mutagenesis techniques set forth herein produce site-directed mutations, sequencing of the altered peptide should not be necessary if the mass spectrograph agrees with prediction. If necessary to verify a changed residue in a protein variant, CAD-tandem MS/MS can be employed to sequence the peptides of the mixture in question, or the target peptide can be purified for subtractive Edman degradation or carboxypeptidase Y digestion depending on the location of the modification.

Other site directed mutagenesis techniques may also be employed with the nucleotide sequences of the invention. For example, restriction endonuclease digestion of DNA followed by ligation may be used to generate deletion variants of proteins of the present invention, as described in section 15.3 of Sambrook et al. Molecular Cloning: A Laboratory Manual, 2nd Ed., Cold Spring Harbor Laboratory Press, New York, N.Y. (1989), incorporated herein by reference. A similar strategy may be used to construct insertion variants, as described in section 15.3 of Sambrook et al., supra.

Oligonucleotide-directed mutagenesis may also be employed for preparing substitution variants of this invention. It may also be used to conveniently prepare the deletion and insertion variants of this invention. This technique is well known in the art as described by Adelman et al. (DNA 2:183, 1983); Sambrook et al., supra; Current Protocols in Molecular Biology, 1991, Wiley (NY), F. T. Ausubel et al. eds., incorporated herein by reference.

Generally, oligonucleotides of at least 25 nucleotides in length are used to insert, delete or substitute two or more nucleotides in a nucleic acid molecule encoding a protein of the invention. An optimal oligonucleotide will have 12 to 15 perfectly matched nucleotides on either side of the nucleotides coding for the mutation. To mutagenize a wild-type protein, the oligonucleotide is annealed to the single-stranded DNA template molecule under suitable hybridization conditions. A DNA polymerizing enzyme, usually the Klenow fragment of E. coli DNA polymerase I, is then added. This enzyme uses the oligonucleotide as a primer to complete the synthesis of the mutation-bearing strand of DNA. Thus, a heteroduplex molecule is formed such that one strand of DNA encodes the wild-type synthase inserted in the vector, and the second strand of DNA encodes the mutated form of the synthase inserted into the same vector. This heteroduplex molecule is then transformed into a suitable host cell.

Mutants with more than one amino acid substituted may be generated in one of several ways. If the amino acids are located close together in the polypeptide chain, they may be mutated simultaneously using one oligonucleotide that codes for all of the desired amino acid substitutions. If, however, the amino acids are located some distance from each other (separated by more than ten amino acids, for example) it is more difficult to generate a single oligonucleotide that encodes all of the desired changes. Instead, one of two alternative methods may be employed. In the first method, a separate oligonucleotide is generated for each amino acid to be substituted. The oligonucleotides are then annealed to the single-stranded template DNA simultaneously, and the second strand of DNA that is synthesized from the template will encode all of the desired amino acid substitutions. An alternative method involves two or more rounds of mutagenesis to produce the desired mutant. The first round is as described for the single mutants: DNA encoding wild-type protein is used for the template, an oligonucleotide encoding the first desired amino acid substitution(s) is annealed to this template, and the heteroduplex DNA molecule is then generated. The second round of mutagenesis utilizes the mutated DNA produced in the first round of mutagenesis as the template. Thus, this template already contains one or more mutations. The oligonucleotide encoding the additional desired amino acid substitution(s) is then annealed to this template, and the resulting strand of DNA now encodes mutations from both the first and second rounds of mutagenesis. This resultant DNA can be used as a template in a third round of mutagenesis, and so on.

Eukaryotic expression systems may be utilized for the production of proteins of the invention since they are capable of carrying out any required posttranslational modifications and of directing the proteins to the proper cellular compartment. A representative eukaryotic expression system for this purpose uses the recombinant baculovirus, Autographa californica nuclear polyhedrosis virus (AcNPV; M. D. Summers and G. E. Smith, A Manual of Methods for Baculovirus Vectors and Insect Cell Culture Procedures (1986); Luckow et al., Bio-technology 6:47-55, 1987) for expression of the proteins of the invention. Infection of insect cells (such as cells of the species Spodoptera frugiperda) with the recombinant baculoviruses allows for the production of large amounts of proteins. In addition, the baculovirus system has other important advantages for the production of recombinant proteins. For example, baculoviruses do not infect humans and can therefore be safely handled in large quantities. In the baculovirus system, a DNA construct is prepared including a vector and a DNA segment encoding a protein. The vector may comprise the polyhedron gene promoter region of a baculovirus, the baculovirus flanking sequences necessary for proper cross-over during recombination (the flanking sequences comprise about 200-300 base pairs adjacent to the promoter sequence) and a bacterial origin of replication which permits the construct to replicate in bacteria. The vector is constructed so that (i) the DNA segment is placed adjacent (or operably linked or “downstream” or “under the control of”) to the polyhedron gene promoter and (ii) the promoter/protein combination is flanked on both sides by 200-300 base pairs of baculovirus DNA (the flanking sequences).

To produce the desired DNA construct, a cDNA clone encoding the full length protein is obtained using methods such as those described herein. The DNA construct is contacted in a host cell with baculovirus DNA of an appropriate baculovirus (that is, of the same species of baculovirus as the promoter encoded in the construct) under conditions such that recombination is effected. The resulting recombinant baculoviruses encode the full-length protein. For example, an insect host cell can be cotransfected or transfected separately with the DNA construct and a functional baculovirus. Resulting recombinant baculoviruses can then be isolated and used to infect cells to effect production of the protein. Host insect cells include, for example, Spodoptera frugiperda cells, that are capable of producing a baculovirus-expressed protein. Insect host cells infected with a recombinant baculovirus of the present invention are then cultured under conditions allowing expression of the baculovirus-encoded protein. Protein thus produced is then extracted from the cells using methods known in the art.

Other eukaryotic microbes such as yeasts may also be used in the practice of the present invention, for example to express the proteins of the present invention. The baker's yeast Saccharomyces cerevisiae, is a commonly used yeast, although several other strains are available. The plasmid YRp7 (Stinchcomb et al., Nature 282:39, 1979; Kingsman et al., Gene 7:141, 1979; Tschemper et al., Gene 10:157, 1980, is commonly used as an expression vector in Saccharomyces. This plasmid contains the trp1 gene that provides a selection marker for a mutant strain of yeast lacking the ability to grow in tryptophan, such as strains ATCC No. 44,076 and PEP4-1 (Jones, Genetics, 85:12, 1977. The presence of the trp1 lesion as a characteristic of the yeast host cell genome then provides an effective environment for detecting transformation by growth in the absence of tryptophan. Yeast host cells are generally transformed using the polyethylene glycol method, as described by Hinnen (Proc. Natl. Acad. Sci. USA 75:1929, 1978. Additional yeast transformation protocols are set forth in Gietz et al., N.A.R. 20(17):1425, 1992; Reeves et al., FEMS 99(2-3):193-197, 1992, both of which publications are incorporated herein by reference.

Suitable promoting sequences in yeast vectors include the promoters for 3-phosphoglycerate kinase (Hitzeman et al., J. Biol. Chem. 255:2073, 1980 or other glycolytic enzymes (Hess et al., J. Adv. Enzyme Reg. 7:149, 1968; Holland et al., Biochemistry 17:4900, 1978), such as enolase, glyceraldehyde-3-phosphate dehydrogenase, hexokinase, pyruvate decarboxylase, phosphofructokinase, glucose-6-phosphate isomerase, 3-phosphoglycerate mutase, pyruvate kinase, triosephosphate isomerase, phosphoglucose isomerase, and glucokinase. In the construction of suitable expression plasmids, the termination sequences associated with these genes are also ligated into the expression vector 3′ of the sequence desired to be expressed to provide polyadenylation of the mRNA and termination. Other promoters that have the additional advantage of transcription controlled by growth conditions are the promoter region for alcohol dehydrogenase 2, isocytochrome C, acid phosphatase, degradative enzymes associated with nitrogen metabolism, and the aforementioned glyceraldehyde-3-phosphate dehydrogenase, and enzymes responsible for maltose and galactose utilization. Any plasmid vector containing yeast-compatible promoter, origin of replication and termination sequences is suitable.

Cell cultures derived from multicellular organisms, such as plants, may be used as hosts to practice this invention. Transgenic plants can be obtained, for example, by transferring plasmids that encode a protein of the invention and a selectable marker gene, e.g., the kan gene encoding resistance to kanamycin, into Agrobacterium tumifaciens containing a helper Ti plasmid as described in Hoeckema et al., Nature 303:179-181, 1983, and culturing the Agrobacterium cells with leaf slices, or other tissues or cells, of the plant to be transformed as described by An et al., Plant Physiology 81:301-305, 1986. Transformation of cultured plant host cells is normally accomplished through Agrobacterium tumifaciens. Cultures of mammalian host cells and other host cells that do not have rigid cell membrane barriers are usually transformed using the calcium phosphate method as originally described by Graham and Van der Eb (Virology 52:546, 1978) and modified as described in sections 16.32-16.37 of Sambrook et al., supra. However, other methods for introducing DNA into cells such as Polybrene (Kawai and Nishizawa, Mol. Cell. Biol. 4:1172, 1984), protoplast fusion (Schaffner, Proc. Natl. Acad. Sci. USA 77:2163, 1980), electroporation (Neumann et al., EMBO J. 1:841, 1982), and direct microinjection into nuclei (Capecchi, Cell 22:479M 1980) may also be used. Additionally, animal transformation strategies are reviewed in Monastersky G. M. and Robl, J. M., Strategies in Transgenic Animal Science, ASM Press, Washington, D.C., 1995, incorporated herein by reference. Transformed plant calli may be selected through the selectable marker by growing the cells on a medium containing, e.g., kanamycin, and appropriate amounts of phytohormone such as naphthalene acetic acid and benzyladenine for callus and shoot induction. The plant cells may then be regenerated and the resulting plants transferred to soil using techniques well known to those skilled in the art.

In addition, a nucleic acid molecule encoding a protein of the present invention can be incorporated into a plant along with a necessary promoter which is inducible. In the practice of this embodiment of the invention, a promoter that only responds to a specific external or internal stimulus is fused to the target cDNA. Thus, the nucleic acid molecule will not be transcribed except in response to the specific stimulus. As long as the nucleic acid molecule is not being transcribed, its protein product is not produced.

An illustrative example of a responsive promoter system that can be used in the practice of this invention is the glutathione-S-transferase (GST) system in maize. GSTs are a family of enzymes that can detoxify a number of hydrophobic electrophilic compounds that often are used as pre-emergent herbicides (Weigand et al., Plant Molecular Biology 7:235-243, 1986). Studies have shown that the GSTs are directly involved in causing this enhanced herbicide tolerance. This action is primarily mediated through a specific 1.1 kb mRNA transcription product. In short, maize has a naturally occurring quiescent gene already present that can respond to external stimuli and that can be induced to produce a gene product. This gene has previously been identified and cloned. Thus, in one embodiment of this invention, the promoter is removed from the GST responsive gene and attached to a gene of the present invention that previously has had its native promoter removed. This engineered gene is the combination of a promoter that responds to an external chemical stimulus and a gene responsible for successful production of a protein of the present invention.

In addition to the methods described above, several methods are known in the art for transferring cloned DNA into a wide variety of plant species, including gymnosperms, angiosperms, monocots and dicots (see, e.g., Glick and Thompson, eds., Methods in Plant Molecular Biology, CRC Press, Boca Raton, Fla. (1993), incorporated by reference herein). Representative examples include electroporation-facilitated DNA uptake by protoplasts in which an electrical pulse transiently permeabilizes cell membranes, permitting the uptake of a variety of biological molecules, including recombinant DNA (Rhodes et al., Science 240(4849):204-207, 1988); treatment of protoplasts with polyethylene glycol (Lyznik et al., Plant Molecular Biology 13:151-161, 1989); and bombardment of cells with DNA-laden microprojectiles which are propelled by explosive force or compressed gas to penetrate the cell wall (Klein et al., Plant Physiol. 91:440-444, 1989, and Boynton et al., Science 240(4858):1534-1538, 1988). A method that has been applied to Rye plants (Secale cereale) is to directly inject plasmid DNA, including a selectable marker gene, into developing floral tillers (de la Pena et al., Nature 325:274-276, 1987). Further, plant viruses can be used as vectors to transfer genes to plant cells. Examples of plant viruses that can be used as vectors to transform plants include the Cauliflower Mosaic Virus (Brisson et al., Nature 310:511-514, 1984. Additionally, plant transformation strategies and techniques are reviewed in Birch, R. G., Ann. Rev. Plant Phys. Plant Mol. Biol. 48:297, 1997; Forester et al., Exp. Agric. 33:15-33, 1997. The aforementioned publications disclosing plant transformation techniques are incorporated herein by reference, and minor variations make these technologies applicable to a broad range of plant species.

The cells which have been transformed may be grown into plants by a variety of art-recognized means. See, for example, McConnick et al., Plant Cell Reports 5:81-84, 1986. These plants may then be grown, and either selfed or crossed with a different plant strain, and the resulting homozygotes or hybrids having the desired phenotypic characteristic identified. Two or more generations may be grown to ensure that the subject phenotypic characteristic is stably maintained and inherited and then seeds harvested to ensure the desired phenotype or other property has been achieved.

The following are representative plant species that are suitable for genetic manipulation in accordance with the present invention. The citations are to representative publications disclosing genetic transformation protocols that can be used to genetically transform the listed plant species. Each of the following publications relating to plant transformation are incorporated herein by reference. Rice (Alam, M. F. et al., Plant Cell Rep. 18:572-575, 1999); maize (Merlo, A. O. et al., Plant Cell 10:1603-1621, 1998); wheat (Ortiz, J. P. A. et al., Plant Cell Rep. 15:877-881, 1996); tomato (Filatti, J. J. et al., Bio/Technology 5:726-730, 1987); potato (Kumar, A. et al., Plant J. 9:821-829, 1996); cassaya (Li, H.-Q. et al., Nat. Biotechnology, 14:736-740, 1996); lettuce (Michelmore, R. et al., Plant Cell Rep 6:439-442 (1987)); tobacco (Horsch, R. B. et al., Science, 227:1229-1231, 1985); cotton (McCabe, D. E. and Martinell, B. J., Biotechnology 11:596-598, 1993); grasses (Xiao, L. and Ha, S.-B., Plant Cell Rep. 16:874-878 (1997); Ye, X. et al., Plant Cell Rep. 16:379-384, 1997; Dalton, S. J. et al., Plant Sci. 132:31-43, 1998; Hartman, C. L., Lee, L., Day, P. R. and N. E. Turner, Bio/Tech. 12:919-923, 1994; Inokuma, C., Sugiura, K., Imaizumi, N. and C. Cho, Plant Cell Rep. 17:334-338, 1998; Lee, L., Laramore, C. L., Day, P. R. and N. E. Tumer, Crop Sci. 36:401-406, 1996; Spangenberg, G., Wang, Z.-Y., Nagel, J. and I. Potrykus, Plant Sci. 97:83-94, 1994; Spangenberg, G., Wang, Z.-Y., Wu, X, Nagel, J. and I. Potrykus, Plant Sci. 108:209-217, 1995; Takamizo, T., Suginobu, K. and G. Ohsugi, Plant Science 72:125-131, 1990; Wang, G. R., Binding, H. and U. K. Posselt, Plant Physiol. 151:83-90, 1997; Wang, Z. Y., Nagel, J., Potrykus, I. and G. Spangenberg, Plant Sci. 94:179-193, 1993); peppermint (X. Niu et al., Plant Cell Reports 17:165-171, 1998); citrus plants (Pena, L. et al., Plant Science 104:183-191, 1995); caraway (F. A. Krens, et al., Plant Cell Reports 17:39-43, 1997); and Artemisia (S. Banerjee et al., Planta Medica 63(5):467-469, 1997).

Each of these techniques has advantages and disadvantages. In each of the techniques, DNA from a plasmid is genetically engineered such that it contains not only the gene of interest, but also selectable and screenable marker genes. A selectable marker gene is used to select only those cells that have integrated copies of the plasmid (the construction is such that the gene of interest and the selectable and screenable genes are transferred as a unit). The screenable gene provides another check for the successful culturing of only those cells carrying the genes of interest. A commonly used selectable marker gene is neomycin phosphotransferase II (NPT II). This gene conveys resistance to kanamycin, a compound that can be added directly to the growth media on which the cells grow. Plant cells are normally susceptible to kanamycin and, as a result, die. The presence of the NPT II gene overcomes the effects of the kanamycin and each cell with this gene remains viable. Another selectable marker gene which can be employed in the practice of this invention is the gene which confers resistance to the herbicide glufosinate (Basta). A screenable gene commonly used is the β-glucuronidase gene (GUS). The presence of this gene is characterized using a histochemical reaction in which a sample of putatively transformed cells is treated with a GUS assay solution. After an appropriate incubation, the cells containing the GUS gene turn blue.

The plasmid containing one or more of these genes is introduced into either plant protoplasts or callus cells by any of the previously mentioned techniques. If the marker gene is a selectable gene, only those cells that have incorporated the DNA package survive under selection with the appropriate phytotoxic agent. Once the appropriate cells are identified and propagated, plants are regenerated. Progeny from the transformed plants must be tested to insure that the DNA package has been successfully integrated into the plant genome.

Mammalian host cells may also be used in the practice of the invention, for example to express proteins of the present invention. Examples of suitable mammalian cell lines include monkey kidney CVI line transformed by SV40 (COS-7, ATCC CRL 1651); human embryonic kidney line 293S (Graham et al., J. Gen. Virol. 36:59, 1977); baby hamster kidney cells (BHK, ATCC CCL 10); Chinese hamster ovary cells (Urlab and Chasin, Proc. Natl. Acad. Sci USA 77:4216 (1980)); mouse sertoli cells (TM4, Mather, Biol. Reprod. 23:243, 1980); monkey kidney cells (CVI-76, ATCC CCL70); African green monkey kidney cells (VERO-76, ATCC CRL-1587); human cervical carcinoma cells (HELA, ATCC CCL 2); canine kidney cells (MDCK, ATCC CCL 34); buffalo rat liver cells (BRL 3A, ATCC CRL 1442); human lung cells (W138, ATCC CCL 75); human liver cells (Hep G2, HB 8065); mouse mammary tumor cells (MMT 060562, ATCC CCL 51); rat hepatoma cells (HTC, MI.54, Baumann et al., J. Cell Biol. 85:1, 1980); and TR1 cells (Mather et al., Annals N.Y. Acad. Sci. 383:44, 1982). Expression vectors for these cells ordinarily include (if necessary) DNA sequences for an origin of replication, a promoter located in front of the gene to be expressed, a ribosome binding site, an RNA splice site, a polyadenylation site, and a transcription terminator site.

Promoters used in mammalian expression vectors are often of viral origin. These viral promoters are commonly derived from polyoma virus, Adenovirus 2, and most frequently Simian Virus 40 (SV40). The SV40 virus contains two promoters that are termed the early and late promoters. These promoters are particularly useful because they are both easily obtained from the virus as one DNA fragment that also contains the viral origin of replication (Fiers et al., Nature 273:113, 1978). Smaller or larger SV40 DNA fragments may also be used, provided they contain the approximately 250-bp sequence extending from the HindIII site toward the BglI site located in the viral origin of replication.

Alternatively, promoters that are naturally associated with the foreign gene (homologous promoters) may be used provided that they are compatible with the host cell line selected for transformation.

An origin of replication may be obtained from an exogenous source, such as SV40 or other virus (e.g., Polyoma, Adeno, VSV, BPV) and inserted into the cloning vector. Alternatively, the origin of replication may be provided by the host cell chromosomal replication mechanism. If the vector containing the foreign gene is integrated into the host cell chromosome, the latter is often sufficient.

The use of a secondary DNA coding sequence can enhance production levels of recombinant protein in transformed cell lines. The secondary coding sequence typically comprises the enzyme dihydrofolate reductase (DHFR). The wild-type form of DHFR is normally inhibited by the chemical methotrexate (MTX). The level of DHFR expression in a cell will vary depending on the amount of MTX added to the cultured host cells. An additional feature of DHFR that makes it particularly useful as a secondary sequence is that it can be used as a selection marker to identify transformed cells. Two forms of DHFR are available for use as secondary sequences, wild-type DHFR and MTX-resistant DHFR. The type of DHFR used in a particular host cell depends on whether the host cell is DHFR deficient (such that it either produces very low levels of DHFR endogenously, or it does not produce functional DHFR at all). DHFR-deficient cell lines such as the CHO cell line described by Urlaub and Chasin, supra, are transformed with wild-type DHFR coding sequences. After transformation, these DHFR-deficient cell lines express functional DHFR and are capable of growing in a culture medium lacking the nutrients hypoxanthine, glycine and thymidine. Nontransformed cells will not survive in this medium.

The MTX-resistant form of DHFR can be used as a means of selecting for transformed host cells in those host cells that endogenously produce normal amounts of functional DHFR that is MTX sensitive. The CHO-K1 cell line (ATCC No. CL 61) possesses these characteristics, and is thus a useful cell line for this purpose. The addition of MTX to the cell culture medium will permit only those cells transformed with the DNA encoding the MTX-resistant DHFR to grow. The nontransformed cells will be unable to survive in this medium.

Prokaryotes may also be used as host cells for the initial cloning steps of this invention and/or to express the proteins of the invention. They are particularly useful for rapid production of large amounts of DNA, for production of single-stranded DNA templates used for site-directed mutagenesis, for screening many mutants simultaneously, and for DNA sequencing of the mutants generated. Suitable prokaryotic host cells include E. coli K12 strain 94 (ATCC No. 31,446), E. coli strain W3110 (ATCC No. 27,325) E. coli X1776 (ATCC No. 31,537), and E. coli B; however many other strains of E. coli, such as HB101, JM101, NM522, NM538, NM539, and many other species and genera of prokaryotes including bacilli such as Bacillus subtilis, other enterobacteriaceae such as Salmonella typhimurium or Serratia marcesans, and various Pseudomonas species may all be used as hosts. Prokaryotic host cells or other host cells with rigid cell walls are preferably transformed using the calcium chloride method as described in section 1.82 of Sambrook et al., supra. Alternatively, electroporation may be used for transformation of these cells. Prokaryote transformation techniques are set forth in Dower, W. J., in Genetic Engineering, Principles and Methods 12:275-296, Plenum Publishing Corp. (1990); Hanahan et al., Meth. Enzymol. 204:63, 1991.

As a representative example, cDNA sequences encoding proteins of the invention may be transferred to the (His)₆.Tag pET vector commercially available (from Novagen, Madison Wis.) for overexpression in E. coli as heterologous host. This pET expression plasmid has several advantages in high level heterologous expression systems. The desired cDNA insert is ligated in frame to plasmid vector sequences encoding six histidines followed by a highly specific protease recognition site (thrombin) that are joined to the amino terminus codon of the target protein. The histidine “block” of the expressed fusion protein promotes very tight binding to immobilized metal ions and permits rapid purification of the recombinant protein by immobilized metal ion affinity chromatography. The histidine leader sequence is then cleaved at the specific proteolysis site by treatment of the purified protein with thrombin, and the expressed protein again purified by immobilized metal ion affinity chromatography, this time using a shallower imidazole gradient to elute the recombinant synthases while leaving the histidine block still adsorbed. This overexpression-purification system has high capacity, excellent resolving power and is fast, and the chance of a contaminating E. coli protein exhibiting similar binding behavior (before and after thrombin proteolysis) is extremely small.

As will be apparent to those skilled in the art, any plasmid vectors containing replicon and control sequences that are derived from species compatible with the host cell may also be used in the practice of the invention. The vector usually has a replication site, marker genes that provide phenotypic selection in transformed cells, one or more promoters, and a polylinker region containing several restriction sites for insertion of foreign DNA. Plasmids typically used for transformation of E. coli include pBR322, pUC18, pUC19, pUCI18, pUC119, and Bluescript M13, all of which are described in sections 1.12-1.20 of Sambrook et al., supra. However, many other suitable vectors are available as well. These vectors contain genes coding for ampicillin and/or tetracycline resistance which enables cells transformed with these vectors to grow in the presence of these antibiotics.

The promoters most commonly used in prokaryotic vectors include the β-lactamase (penicillinase) and lactose promoter systems (Chang et al. Nature 375:615, 1978; Itakura et al., Science 198:1056, 1977; Goeddel et al., Nature, 281:544, 1979) and a tryptophan (trp) promoter system (Goeddel et al., Nucl. Acids Res. 8:4057, 1980; EPO Appl. Publ. No. 36,776), and the alkaline phosphatase systems. While these are the most commonly used, other microbial promoters have been utilized, and details concerning their nucleotide sequences have been published, enabling a skilled worker to ligate them functionally into plasmid vectors (see Siebenlist et al., Cell 20:269, 1980).

Trafficking sequences from plants, animals and microbes can be employed in the practice of the invention to direct the proteins of the present invention to the cytoplasm, endoplasmic reticulum, mitochondria or other cellular components, or to target the protein for export to the medium. Many eukaryotic proteins normally secreted from the cell contain an endogenous secretion signal sequence as part of the amino acid sequence. Thus, proteins normally found in the cytoplasm can be targeted for secretion by linking a signal sequence to the protein. This is readily accomplished by ligating DNA encoding a signal sequence to the 5′ end of the DNA encoding the protein and then expressing this fusion protein in an appropriate host cell. The DNA encoding the signal sequence may be obtained as a restriction fragment from any gene encoding a protein with a signal sequence. Thus, prokaryotic, yeast, and eukaryotic signal sequences may be used herein, depending on the type of host cell utilized to practice the invention. The DNA and amino acid sequence encoding the signal sequence portion of several eukaryotic genes including, for example, human growth hormone, proinsulin, and proalbumin are known (see Stryer, Biochemistry W.H. Freeman and Company, New York, N.Y., p. 769 (1988)), and can be used as signal sequences in appropriate eukaryotic host cells. Yeast signal sequences, as for example acid phosphatase (Arima et al., Nuc. Acids Res. 11:1657, 1983), α-factor, alkaline phosphatase and invertase may be used to direct secretion from yeast host cells. Prokaryotic signal sequences from genes encoding, for example, LamB or OmpF (Wong et al., Gene 68:193, 1988), MalE, PhoA, or beta-lactamase, as well as other genes, may be used to target proteins from prokaryotic cells into the culture medium.

The construction of suitable vectors containing DNA encoding replication sequences, regulatory sequences, phenotypic selection genes and the DNA of interest are prepared using standard recombinant DNA procedures. Isolated plasmids and DNA fragments are cleaved, tailored, and ligated together in a specific order to generate the desired vectors, as is well known in the art (see, for example, Sambrook et al., supra).

The nucleic acid molecules of the present invention, such as the nucleic acid molecules having the sequences set forth in SEQ ID NOS:1-472 can also be used to generate probes for mapping the genome of plant species such as the peppermint plant (Mentha piperita) and its relatives. The probe may be mapped to a particular chromosome or to a specific region of a chromosome using well known techniques. These include in situ hybridization to chromosomal spreads, flow-sorted chromosomal preparations, or artificial chromosome constructions such as yeast artificial chromosomes (YACs), bacterial artificial chromosomes (BACs), bacterial PI constructions or single chromosome cDNA libraries.

In situ hybridization of chromosomal preparations and physical mapping techniques such as linkage analysis using established chromosomal markers are useful in extending genetic maps. Often the placement of a gene on the chromosome of another species may reveal associated markers. New partial nucleotide sequences can be assigned to chromosomal arms, or parts thereof, by physical mapping. This provides valuable information to investigators searching, for example, for plant disease genes using positional cloning or other gene discovery techniques. Once a plant disease has been localized by genetic linkage to a particular genomic region, any sequences mapping to that area may represent genes for further investigation. The nucleotide sequences of the subject invention may also be used to detect differences in the chromosomal location of nucleotide sequences due to such events as translocation and inversion.

In one representative approach for constructing a physical map of a plant genome, such as the peppermint plant genome, using the nucleic acid molecules of the present invention, genomic DNA is isolated from the plant species of interest and cleaved with one or more restriction enzymes. The resulting fragments are then cloned and mapped as follows. The first stage of the procedure involves a “fingerprinting” procedure for the identification of overlaps between clones. Clones are picked at random, fingerprinted and assembled into overlapping sets referred to as contigs. In the second stage, clones are selected by hybridization using probes from the ends of contigs, unattached clones and yeast artificial chromosome (YAC) libraries to fill in the gaps.

The fingerprints are generated by digesting randomly selected clones from primary libraries with one to several restriction enzymes. Following size fractionation by gel electrophoresis (either agarose or polyacrylamide), the lengths of the fragments are determined. The number and the size of the fragments constitute a unique signature or fingerprint of the cloned insert. For fingerprinting, it is unnecessary to generate a restriction map of the clone. The bands must however be descriptive of the insert and the informational content of the fingerprint must be sufficient to make a reliable assignment of overlapping regions. Clones are said to be overlapping when the fingerprints of two clones are sufficiently similar.

The fingerprinting protocols described herein are based on the methodologies of Coulson, A. et al. “Toward a physical map of the nematode Caenorhabditis elegans” Proc Natl Acad. Sci. USA 83:7821-7825, 1986. In brief, cloned DNAs are digested with a restriction enzyme having a 6 bp specificity which leaves staggered ends which are simultaneously labeled with reverse transcriptase and the appropriate nucleoside triphosphates. The reactions are terminated by high temperature and the fragments are subjected to a second round of cleavage with a restriction enzyme having a 4 bp specificity. The resultant fragments are size-fractionated, for example on a denaturing 4% polyacrylamide gel. The positions of the bands are typically entered into a computer using a scanning densitometer and an image-processing package, such as those described in Sulston et al. “Software for genome mapping by fingerprinting techniques” Comput. Applic. Biosci. 4:125-132, 1988; Sulston et al. “Image analysis of restriction enzyme fingerprint autoradiograms” Computer Applic. BioSci. 5:101-106, 1989, both of which publications are incorporated herein by reference.

Once the banding patterns of individual clones are entered into the computer, or are otherwise recorded, they are then compared in a pairwise fashion against the entire data set. The output is a ranked order of the most probable matches. Based on these numbers, the regions of probable overlap are determined and the clones are assembled into contigs, for example by using the computer program disclosed in Coulson et al. “Toward a physical map of the nematode Caenorhabditis elegans” Proc Natl Acad. Sci USA 83:7821-7825, 1986. Before the clones are joined, the reliability of the match is assessed by visually aligning the films and the overlap must be logically consistent. Although the use of computers greatly facilitates the comparison of restriction pattern “fingerprints”, especially when the investigator is comparing the “fingerprints” of a large number of clones, the comparison can be done manually by visually comparing the “fingerprints” of individual clones.

One of the main considerations for choosing an enzyme or combination of enzymes is that the number of fragments generated is optimal for the statistical detection of overlapping regions. Preferably, it is desirable to use several combinations of enzymes because it is unlikely that a given clone will have a non-random distribution of restriction sites for all of the chosen enzymes.

By increasing the amount of information obtained from each clone, the rate of progress is greatly increased. A mathematical analysis of random clone fingerprinting by Lander and Waterman “Genomic mapping by fingerprinting random clones: a mathematical analysis” Genomics 2:231-239, 1988, shows that decreasing the minimal detectable overlap from 50 to 25% significantly speeds the progress of a project. Based on this analysis, it is desirable to use fingerprinting strategies which detect overlaps in the range of 15 to 20%.

In general, 8 to 10 genomic equivalents must be fingerprinted to achieve between 70 and 90% coverage of the genome. It is desirable, therefore, to automate as many steps of the process as possible, such as by the use of automatic data collection (see, e.g., Brenner and Livik “DNA fingerprinting by sampled sequencing” Proc Natl Acad. Sci USA 86:8902-8906, 1989; Carrano et al. “A high-resolution fluorescence-based, semiautomated method for DNA fingerprinting” Genomics 4:129-136, 1989) and by the use of commercially available automated DNA sequencers (Smith, L. M. et al. “Fluorescence detection in automated DNA sequence analysis” Nature 321:674-679, 1986).

Random fingerprinting procedures are not expected to produce complete physical maps. Instead, the map will consist of many contigs composed of two or more overlapping clones. As the project progresses, the number of contigs decreases as the gaps are closed. After this point, the rate of finding new contigs significantly decreases due to the scarcity of the remaining clones. Completion of the map then requires a directed approach since a prohibitively large number of clones would be required to close all of the gaps by random clone fingerprinting.

In addition to the statistical limitations, both the number and the size of the contigs generated by random clone mapping will be strongly influenced by any cloning biases which are encountered. At least two factors contribute to cloning bias: the inability to clone certain regions of the genome using a given host/vector system results in non-representative libraries and non-uniform growth of individual clones leading to sampling bias. To circumvent such problems, it is likely that multiple libraries and multiple host vector systems will be required.

Once the practical limit of random clone mapping is reached, success in completing a map depends largely on the ability to bridge the remaining gaps. The most viable option is to select the missing clones by hybridization. One approach for selecting linking clones is to make end-probes from unattached clones (ie., clones that have not yet been incorporated into the map) and clones residing at the end of the contigs. This approach is facilitated if it is possible to generate end-probes with minimal effort. The cosmid libraries are therefore preferably constructed in vectors containing convergent bacteriophage promoters (for example Sp6 and T7 promoters) flanking the insert. The end-clones and the unattached clones are picked into microtiter dishes and plated out onto nylon filters in ordered arrays. By probing the cosmid grids with mixed RNA probes (prepared from rows of clones) (Evans and Lewis “Physical mapping of complex genomes by cosmid multiplex analysis” Proc Natl Acad. Sci USA 86:5030-5034, 1989), overlaps which were not detected by fingerprint analysis can be established. The use of mixed end-probes is important when a large number of joins must be established since the number of hybridizations required is reduced by a factor of N, where N is the number of clones used to make the probes.

Missing clones may be either rare or non-existent in the cosmid libraries which were used for the random clone mapping. Therefore, the end-probes can also be used to probe additional libraries based on different host/vector systems. The use of different host/vector systems is intended to eliminate, or at least reduce, cloning bias. In particular, the hybridization to yeast artificial chromosome (YAC) clones is an important component for this analysis.

One of the most important new technical advances in molecular biology is the cloning of megabase-size DNA fragments using YAC vectors (Burke et al. “Cloning of large segments of exogenous DNA into yeast by means of artificial chromosome vectors” Science 236:806-812, 1987). The construction of YAC libraries involves the ligation of large DNA fragments (50-1000 kb) into a vector containing selectable markers and the functional components of a eukaryotic chromosome, i.e., ARS elements required for autonomous replication, the centromere which results in proper disjunction during meiosis and mitosis, and telomeres required for the replication of linear molecules (Murry and Szostak, “Construction of artificial chromosomes in yeast” Nature 305:189-193, 1983). The constructs are transformed into Saccharomyces cerevisiae where they are replicated along with the endogenous chromosomes. The large size of YAC clones means that fewer clones must be examined, and YACs offer the potential to give a random or at least different representation of clones than are obtained using bacterial host/vector systems.

A complementary approach to bridge the gaps is to use YAC clones as hybridization probes (Coulson et al. “Genome linking with yeast artificial chromosomes” Nature 335:184-186, 1988). The strategy is to prepare two sets of ordered grids: one of a representative YAC library and one of cosmids which is as representative as possible of both the contigs and unattached clones. The YACs are then separated from the host chromosomes by electrophoresis, isolated from the gel and used to make hybridization probes. The hybridization pattern of the cosmid grid is then used to establish linkage as well as the position of the YAC with respect to the ordered cosmids. Since a given YAC clone is expected to hybridize to several clones in the contig, the hybridization patterns must conform to the logic of the contig map thereby minimizing spurious linkage resulting from hybridization to interspersed repeats.

The YACs to be used as probes may be picked at random or, alternatively, selected from the YAC grid based on hybridization with cosmids as described above. One requirement of this approach is that the cosmid vectors have no significant homology to the YAC vectors. This permits the direct hybridization of the YACs to cosmids, and vice versa, thereby eliminating the need to first separate the insert from the vector sequences. The Lorist (Cross and Little, “A cosmid vector for systematic chromosome walking” Gene 49:9-22, 1986) series of cosmid vectors have been successfully used for this approach (Coulson et al. “Genome linking with yeast artificial chromosomes” Nature 335:184-186, 1988).

YAC clones may be used at the onset of physical mapping projects. Using existing technology it is possible to fingerprint YACs directly (Kuspa et al. “Physical mapping of the Myxococcus xanthus genome by random cloning in yeast artificial chromosomes” Proc Natl Acad. Sci USA 86:8917-8921, 1989). Moreover, the ability to easily generate end-probes from YACs using techniques such as inverse PCR (Ochman et al. “Genetic applications of an inverse polymerase chain reaction” Genetics 120:621-623, 1988) allows for the construction of physical maps based on hybridization strategies. It is unlikely, however, that YACs will supersede cosmid and λ clone maps since the smaller clones are generally required for routine procedures such as DNA sequencing and gene isolation.

The resulting physical map of a plant genome (such as the genome of the peppermint plant) is made up of numerous, overlapping DNA fragments and includes the location of restriction enzyme cleavage sites. One way to determine the position of genes of the present invention on the map is to use full-length, or partial length, cDNAs of the invention as probes with which to screen the individual, cloned genomic DNA fragments that were used to construct the map. Thus, for example, individual genomic clones can be digested with one or more restriction enzymes and the digestion products separated on an agarose gel by electrophoresis. The gel can be blotted and probed with radiolabelled cDNA molecules, for example utilizing the hybridization protocol set forth in Example 2 herein. In this way, the location of genes of the present invention (encoding one or more cDNAs of the invention) can be located on the plant genome physical map.

In accordance with the foregoing discussion of plant genome mapping, a representative protocol for physically mapping a plant genome is set forth in Example 5 herein.

The following examples merely illustrate the best mode now contemplated for practicing the invention, but should not be construed to limit the invention.

### EXAMPLE 1

### Construction and Analysis of a Peppermint Oil Gland cDNA Library

mRNA Isolation and cDNA Synthesis: A previously developed method for the isolation of mint oil glands (Gershenzon et al., Recent Adv. Phytochem. 25:347, 1991; Gershenzon et al., Anal. Biochem. 22:130, 1992), that was designed for pathway studies and protein isolation, was not suitable for the isolation of mRNA because of enzymatic and non-enzymatic degradation of nucleic acids (the unmodified protocol yielded no detectable, intact mRNA). Therefore, based upon systematic evaluation of RNA yield and quality by formaldehyde-agarose gel electrophoresis and in vitro translation using the wheat germ system (Titus, Promega Protocols and Application Guide, 2nd ed. (1991)), and by SDS-PAGE of the resulting proteins, the peppermint oil gland secretory cell RNA isolation protocol was modified and then optimized to prevent enzymatic and non-enzymatic degradation of RNA by the addition of 5 mM aurintricarboxylic acid (Gonzalez et al., Biochemistry 19:4299, 1980) and 1 mM thiourea (Van Driesscke et al., Anal. Biochem. 141:184, 19841) to the leaf inhibition solution and buffers utilized.

The resulting peppermint oil gland secretory cells, obtained by this new procedure, were frozen in liquid N₂, powdered with a mortar and pestle, and the RNA was extracted and isolated using a modification of the method of Logemann et al. (Anal. Biochem. 163:16, 1987). This altered protocol involves extraction with 8 M guanidine-HCl and then chloroform-phenol, followed by acid partitioning of DNA into the organic phase and ethanol (10% v/v) precipitation of polysaccharides, prior to precipitation of RNA, and was further modified by the addition of polyvinylpolypyrrolidone to the extraction buffer (Lewinsohn et al., Plant Mol. Biol. Rep. 12:20, 1994) to bind deleterious phenolic materials released during initial disruption of the purified gland cells.

mRNA was isolated by two rounds of oligo(dT)-cellulose column chromatography (Pharmacia Biotech), and the quality was assessed by in vitro translation. mRNA was isolated as set forth in Lewinsohn et al., Plant Molecular Biology Reporter 12(1):20-25, 1994, as modified by homogenization of the plant tissue in the presence of guanidine hydrochloride as set forth in Logemann et al., Analytical Biochemistry 163:16-20, 1987. Typically, 1 g of peppermint oil gland cells yields 0.5-1.0 mg of total RNA from which 1-2% of good quality poly(A)⁺ RNA can be isolated. cDNA synthesis from 5 μg purified mRNA and construction of the λZAPII cDNA expression library were carried out with a commercial kit (Stratagene, La Jolla, Calif.).

DNA Sequencing: The cDNA clones were excised as Bluescript SK (−) phagemids in the bacterial host strain SOLR (Stratagene, La Jolla, Calif.) according to the in vivo excision protocol supplied by Stratagene. Aliquots of the library were plated onto Luria Bertani agar containing 100 μg/ml ampicillin. Single colonies were randomly picked and grown at 37° C. in 4 ml cultures. Plasmid DNA was extracted using the QIAwell 8 Plus Plasmid Kit from Qiagen (Valencia, Calif.), and Taq polymerase cycle sequencing reactions were performed using DyeTerminator Cycle Sequence Ready Reaction with AmpliTaq FS (Catalogue No. 402122, Perkin Elmer, Norwalk, Conn.) and T3 primer. For automated sequence analysis, a model 373 sequencer (Applied Biosystems) was used.

Sequence Analysis And Functional Assignment: Sequences were edited manually to remove contaminants originating from the vector and to discard poor quality 3′ sequence. Sequence comparisons against the genBank non-redundant protein database were performed using the BLASTX algorithm (Altschul et al., J. Mol. Biol. 215:403, 1990). A match was declared when the score was higher than 120 (optimized similarity score), with 65% sequence identity over a minimum of 30 deduced amino acid residues. Sequences were then grouped, where appropriate, into sequence clusters using the TIGR assembler (Sutton et al., Genome Sci. Technol., 1:9-19, 1995). In addition, the sequences of each overlapping fragment were aligned using the fragment assembly program of the Wisconsin Sequence Analysis Package 9 (Genetics Computer Group, Wisconsin; based on the method of Staden (Nucl. Acids Res. 8:3673, 1980)), and consensus sequences were generated with 90% identity over a minimum of 40 nucleotides. Uppercase bases were used where that base occurs in greater than two-thirds of the aligned sequences.

Assignment of Putative Function: The cDNA molecules isolated from the peppermint oil gland cDNA library were grouped into six groups. A first group includes cDNAs encoding proteins that may be involved in the deoxyxylulose-5-phosphate pathway which produces isopentenyl diphosphate (IPP) as the central precursor of terpenoid essential oils. Table 1 identifies members of the first group of nucleic acid molecules of the present invention. The sequences included in Table 1 (and in subsequent Tables 2-5) are set forth in the sequence listing. As used in the sequence listing, the letter “n” or “N” represents an unknown nucleotide, i.e., sequencing of the cDNA molecule did not unambiguously identify the nucleotide represented by the letter “n” or “N”.

TABLE 1
PUTATIVE PROTEINS OF THE
DEOXYXYLULOSE-5-PHOSPHATE PATHWAY

| | |
|---|---|
| | SEQUENCE |
| FUNCTIONAL ASSIGNMENT | IDENTIFIER |

| | | | |
|---|---|---|---|
| 1. | Aldo-Keto Reductase Homologs | | |
| 1.1 | AKR 1 | ML 444 | SEQ ID NO: 1 |
| 1.2 | AKR 2 | ML 437 | SEQ ID NO: 2 |
| 2. | Putative Kinase | ML 100 | SEQ ID NO: 3 |

A second group of sequences includes terpene synthases, a selection of oxidoreductases, cytochrome P₄₅₀-dependent oxidoreductases, putative acyltransferases and putative glucosyltransferases which are likely involved in secondary transformation reactions leading to the terpenoid end products of mint essential oils. Table 2 identifies members of the second group of nucleic acid molecules of the present invention.

TABLE 2
GROUP 2: TERPENE METABOLISM

| | | SEQUENCE | |
|---|---|---|---|
| FUNCTIONAL ASSIGNMENT | | IDENTIFIER | |
| 1. | Terpene Synthases | | |
| 1.1 | Monoterpene Synthases | | |
| 1.1.1 | MS 1 | ML 1128 | SEQ ID NO: 4 |
| 1.1.2 | MS 2 | ML 945 | SEQ ID NO: 5 |
| 1.1.3 | MS 3 | ML 988 | SEQ ID NO: 6 |
| 1.1.4 | MS 4 | ML 127 | SEQ ID NO: 7 |
| 1.1.5 | MS 5 | ML 343 | SEQ ID NO: 8 |
| 1.1.6 | MS 6 | ML 465 | SEQ ID NO: 9 |
| 1.2 | Sesquiterpene Synthases | | |
| 1.2.1 | SS 1 | ML 747 | SEQ ID NO: 10 |
| 1.2.2 | SS 2 | ML 515 | SEQ ID NO: 11 |
| 1.2.3 | SS 3 | ML 757 | SEQ ID NO: 12 |
| 1.2.4 | SS 4 | ML 129 | SEQ ID NO: 13 |
| 1.3 | Diterpene Synthases | | |
| 1.3.1 | DS 1 | ML 1426 | SEQ ID NO: 14 |
| 1.3.2 | DS 2 | ML 458 | SEQ ID NO: 15 |
| 1.3.3 | DS 3 | ML 533 | SEQ ID NO: 16 |
| 2. | Oxidoreductases | | |
| 2.1 | Carbonyl Reductase Homologs | | |
| 2.1.1 | CR 1 | ML 840 | SEQ ID NO: 17 |
| 2.1.2 | CR 2 | ML 472 | SEQ ID NO: 18 |
| 2.2 | NADPH-Dependent Reductase | | |
| | Homologs | | |
| 2.2.1 | NDR 1 | ML 104 | SEQ ID NO: 19 |
| 2.2.2 | NDR 2 | ML 186 | SEQ ID NO: 20 |
| 2.3 | NADPH-Dependent | | |
| | Oxidoreductase (zeta-cryst.) | | |
| 2.3.1 | NDO 1 | ML 665 | SEQ ID NO: 21 |
| 2.3.2 | NDO 2 | ML 503 | SEQ ID NO: 22 |
| 2.3.3 | NDO 3 | ML 1035 | SEQ ID NO: 23 |
| 2.3.4 | NDO 4 | ML 1251 | SEQ ID NO: 24 |
| 2.3.5 | NDO 5 | ML 1377 | SEQ ID NO: 25 |
| 2.3.6 | NDO 6 | ML 194 | SEQ ID NO: 26 |
| 2.3.7 | NDO 7 | ML 766 | SEQ ID NO: 27 |
| 2.4 | Alcohol Dehydrogenase | | |
| | Homologs | | |
| 2.4.1 | ADH 1 | ML 1026 | SEQ ID NO: 28 |
| 2.4.2 | ADH 2 | ML 417 | SEQ ID NO: 29 |
| 2.4.3 | ADH 3 | ML 524 | SEQ ID NO: 30 |
| 2.4.4 | ADH 4 | ML 541 | SEQ ID NO: 31 |
| 2.5 | NADH Ubiquinone | | |
| | Oxidoreductase Homologs | | |
| 2.5.1 | NUO 1 | ML 742 | SEQ ID NO: 32 |
| 2.5.2 | NUO 2 | ML 234 | SEQ ID NO: 33 |
| 2.5.3 | NUO 3 | ML 365 | SEQ ID NO: 34 |
| 2.5.4 | NUO 4 | ML 68 | SEQ ID NO: 35 |
| 2.6 | Epoxide Hydrolase Homologs | | |
| 2.6.1 | EH 1 | ML 212 | SEQ ID NO: 36 |
| 2.6.2 | EH 2 | ML 1211 | SEQ ID NO: 37 |
| 2.7 | NADH Dehydrogenase | | |
| | Homologs | | |
| 2.7.1 | NDH 1 | ML 1106 | SEQ ID NO: 38 |
| 2.7.2 | NDH 2 | ML 1369 | SEQ ID NO: 39 |
| 2.7.3 | NDH 3 | ML 957 | SEQ ID NO: 40 |
| 2.8 | Aldehyde Dehydrogenase | | |
| | Homolog | | |
| 2.8.1 | ALDDH 1 | ML 1108 | SEQ ID NO: 41 |
| 2.9 | Oxidoreductase Homologs | | |
| 2.9.1 | OXRED 1 | ML 167 | SEQ ID NO: 42 |
| 2.9.2 | OXRED 2 | ML 334 | SEQ ID NO: 43 |
| 2.9.3 | OXRED 3 | ML 438 | SEQ ID NO: 44 |
| 2.9.4 | OXRED 4 | MW 348 | SEQ ID NO: 45 |
| 2.9.5 | OXRED 5 | ML 383 | SEQ ID NO: 46 |
| 2.10 | Ribitol Dehydrogenase | | |
| | Homologs | | |
| 2.10.1 | RDH 1 | ML 347 | SEQ ID NO: 47 |
| 2.11 | Mandelonitrile Lyase Homologs | | |
| 2.11.1 | MNL 1 | ML 875 | SEQ ID NO: 48 |
| 2.11.2 | MNL 2 | ML 504 | SEQ ID NO: 49 |
| 3. | Cytochrome P₄₅₀-Dependent | | |
| | Oxidoreductases | | |
| 3.1 | Soybean Cytochrome | | |
| | P₄₅₀ Homologs | | |
| 3.1.1 | CYT 1 | ML 1132 | SEQ ID NO: 50 |
| 3.1.2 | CYT 2 | ML 1374 | SEQ ID NO: 51 |
| 3.1.3 | CYT 3 | ML 139 | SEQ ID NO: 52 |
| 3.1.4 | CYT 4 | ML 272 | SEQ ID NO: 53 |
| 3.1.5 | CYT 5 | ML 868 | SEQ ID NO: 54 |
| 3.1.6 | CYT 6 | ML 962 | SEQ ID NO: 55 |
| 3.2 | Nepeta Cytochrome | | |
| | P₄₅₀ Homologs | | |
| 3.2.1 | CYT 7 | ML 196 | SEQ ID NO: 56 |
| 3.2.2 | CYT 8 | ML 277 | SEQ ID NO: 57 |
| 3.2.3 | CYT 9 | ML 367 | SEQ ID NO: 58 |
| 3.2.4 | CYT 10 | ML 397 | SEQ ID NO: 59 |
| 3.2.5 | CYT 11 | MW 326 | SEQ ID NO: 60 |
| 3.3 | Arabidopsis Cytochrome | | |
| | P₄₅₀ Homologs | | |
| 3.3.1 | CYT 12 | ML 273 | SEQ ID NO: 61 |
| 3.3.2 | CYT 13 | MW 372 | SEQ ID NO: 62 |
| 3.4 | Mentha Cytochrome | | |
| | P₄₅₀ Homologs | | |
| 3.4.1 | CYT 14 | ML 307 | SEQ ID NO: 63 |
| 3.4.2 | CYT 15 | ML 1425 | SEQ ID NO: 64 |
| 3.5 | Solanum Cytochrome | | |
| | P₄₅₀ Homologs | | |
| 3.5.1 | CYT 16 | ML 857 | SEQ ID NO: 65 |
| 4. | Putative Acyltransferases | | |
| | (BEAT Homologs) | | |
| 4.1 | AT 1 | ML 1304 | SEQ ID NO: 66 |
| 4.2 | AT 2 | ML 774 | SEQ ID NO: 67 |
| 5. | Putative Glucosyltransferases | | |
| 5.1 | GT 1 | ML 970 | SEQ ID NO: 68 |
| 5.2 | GT 2 | ML 197 | SEQ ID NO: 69 |
| 5.3 | GT 3 | ML 1163 | SEQ ID NO: 70 |
| 5.4 | GT 4 | ML 772 | SEQ ID NO: 71 |

A third group of sequences includes cDNAs encoding transcription factors and other regulatory proteins, which may be part of the developmental and biosynthetic machinery of oil glands. Table 3 identifies members of the third group of nucleic acid molecules of the present invention.

TABLE 3
TRANSCRIPTION FACTORS AND
REGULATORY PROTEINS

| | | SEQUENCE | |
|---|---|---|---|
| FUNCTIONAL ASSIGNMENT | | IDENTIFIER | |
| 1. | CA 150 Homolog | ML 778 | SEQ ID NO: 72 |
| 2. | CREB-Binding Homolog | ML 1040 | SEQ ID NO: 73 |
| 3. | BRAHMA Homolog | ML 141 | SEQ ID NO: 74 |
| 4. | Homeobox Protein Homolog | ML 163 | SEQ ID NO: 75 |
| 5. | MADS Box Homologs | | |
| 5.1 | MB 1 | ML 1145 | SEQ ID NO: 76 |
| 5.2 | MB 2 | ML 1311 | SEQ ID NO: 77 |
| 6. | b-ZIP Homolog | ML 1205 | SEQ ID NO: 78 |
| 7. | ZTP 3-3 Homolog | ML 346 | SEQ ID NO: 79 |
| 8. | CPM 10 (MYB) Homolog | ML 407 | SEQ ID NO: 80 |
| 9. | APETALA 2 Homolog | ML 929 | SEQ ID NO: 81 |
| 10. | ALY (Coactivator) Homolog | ML 978 | SEQ ID NO: 82 |
| 11. | ELONGATED HYPOCOTYL | ML 1004 | SEQ ID NO: 83 |
| | Homolog | | |
| 12. | Transcription Factor Homolog | ML 1023 | SEQ ID NO: 84 |
| | (AC005397) | | |
| 13. | Transcription Factor Homolog | ML 921 | SEQ ID NO: 85 |
| | (AL031824) | | |
| 14. | Ring H₂ Zink-Finger Homologs | | |
| 14.1 | ZF 1 | ML 512 | SEQ ID NO: 86 |
| 14.2 | ZF 2 | ML 1057 | SEQ ID NO: 87 |
| 15. | Transcription Factor Homolog | ML 1107 | SEQ ID NO: 88 |
| | (X97907) | | |
| 16. | Ethylene-Induced DNA Binding | ML 951 | SEQ ID NO: 89 |
| | Protein | | |
| | Homolog | | |
| 17. | LETHAL LEAF SPOT Homolog | ML 1323 | SEQ ID NO: 90 |
| 18. | LYT B Homologs | | |
| 18.1 | LYTB 1 | ML 320 | SEQ ID NO: 91 |
| 18.2 | LYTB 2 | ML 78 | SEQ ID NO: 92 |
| 18.3 | LYTB 3 | ML 433 | SEQ ID NO: 93 |
| 18.4 | LYTB 4 | ML 70 | SEQ ID NO: 94 |
| 19. | Myb-Related Transcription Factor | ML 160 | SEQ ID NO: 95 |
| | Homolog | | |
| 20. | Homeodomain-Like Protein | ML 1407 | SEQ ID NO: 96 |
| | Homolog | | |
| 21. | P Transcription Factor Homolog | ML 247 | SEQ ID NO: 97 |
| 22. | 14-3-3 G-Box Factor Homolog | ML 684 | SEQ ID NO: 98 |
| 23. | COM AB Homolog | ML 987 | SEQ ID NO: 99 |

A fourth group of sequences includes cDNAs encoding enzymes that may be involved in signal transduction and transport processes occurring during the trafficking and secretion of terpenoid essential oils in glandular trichomes. Table 4 identifies members of the fourth group of nucleic acid molecules of the present invention.

TABLE 4
TRANSPORT AND SIGNAL TRANSDUCTION

| | | SEQUENCE | |
|---|---|---|---|
| FUNCTIONAL ASSIGNMENT | | IDENTIFIER | |
| 1. | Progesterone Binding Protein | | |
| | Homologs | | |
| 1.1 | PBP 1 | ML 1292 | SEQ ID NO: 100 |
| 1.2 | PBP 2 | ML 584 | SEQ ID NO: 101 |
| 1.3 | PBP 3 | ML 1359 | SEQ ID NO: 102 |
| 1.4 | PBP 4 | ML 590 | SEQ ID NO: 103 |
| 2. | ST12P Homolog | ML 124 | SEQ ID NO: 104 |
| 3. | Probable Sugar Carrier Protein | ML 137 | SEQ ID NO: 105 |
| | Homolog | | |
| 4. | Probable Hexose Carrier Protein | ML 692 | SEQ ID NO: 106 |
| | Homolog | | |
| 5. | ABC Transporter Homolog | ML 767 | SEQ ID NO: 107 |
| 6. | Probable Transporter Protein | ML 1016 | SEQ ID NO: 108 |
| | Homolog | | |
| 7. | Sec13 Protein tTransport Protein | ML 1025 | SEQ ID NO: 109 |
| | Homolog | | |
| 8. | Secretory Carrier Membrane | ML 332 | SEQ ID NO: 110 |
| | Protein Homolog | | |
| 9. | Putative White Protein Homologs | | |
| 9.1 | WP 1 | ML 593 | SEQ ID NO: 111 |
| 9.2 | WP 2 | ML 1253 | SEQ ID NO: 112 |
| 10. | Putative Receptor Homolog | ML 86 | SEQ ID NO: 113 |
| 11. | B2 Protein Homolog | ML 245 | SEQ ID NO: 114 |
| 12. | Protein Transport Protein Homolog | MW 360 | SEQ ID NO: 115 |
| 13. | 33 kDa Putative Secretory Protein | ML 853 | SEQ ID NO: 116 |
| | Homolog | | |
| 14. | Putative Transport Inhibitor | | |
| | Response Protein Homologs | | |
| 14.1 | TIRP 1 | ML 166 | SEQ ID NO: 117 |
| 14.2 | TIRP 2 | ML 850 | SEQ ID NO: 118 |

A fifth group of nucleic acid molecules of the present invention includes DNA sequences that encode portions of proteins of diverse, putative function. Table 5 identifies members of the fifth group of nucleic acid molecules of the present invention.

TABLE 5

| FUNCTIONAL ASSIGNMENT | SEQUENCE IDENTIFIER | |
|---|---|---|
| aspartate aminotransferase | mw378.dat | SEQ ID NO: 119 |
| serine hydroxymethyltransferase | ml1247.con | SEQ ID NO: 120 |
| | ml399.con | SEQ ID NO: 121 |
| ferredoxin-like protein | ml464.dat | SEQ ID NO: 122 |
| Thioredoxin-like proteins | ml1047.con | SEQ ID NO: 123 |
| | ml185.con | SEQ ID NO: 124 |
| | mw322.con | SEQ ID NO: 125 |
| Glutaredoxin-like proteins | ml1100.dat | SEQ ID NO: 126 |
| | ml1295.dat | SEQ ID NO: 127 |
| Water stress-inducible protein | mw330.dat | SEQ ID NO: 128 |
| | ml1414.dat | SEQ ID NO: 129 |
| Apospory-related protein | ml144.dat | SEQ ID NO: 130 |
| Auxin-repressed protein | ml388.dat | SEQ ID NO: 131 |
| pop3 peptide homolog | ml598.dat | SEQ ID NO: 132 |
| | ml1202.da | SEQ ID NO: 133 |
| | ml1237.dat | SEQ ID NO: 134 |
| Aluminum-induced protein | ml268.dat | SEQ ID NO: 135 |
| Drought-induced protein | ml542.dat | SEQ ID NO: 136 |
| Hypersensitivity-related protein | ml573.dat | SEQ ID NO: 137 |
| SRC 1 Homolog | ml648.dat | SEQ ID NO: 138 |
| | ml1234.dat | SEQ ID NO: 139 |
| Cold acclimation protein | ml728.dat | SEQ ID NO: 140 |
| Putative argonaute protein | ml887.dat | SEQ ID NO: 141 |
| Symbiosis-related protein | ml467.dat | SEQ ID NO: 142 |
| | ml1313.dat | SEQ ID NO: 143 |
| Photoassimilate-responsive protein | ml1338.dat | SEQ ID NO: 144 |
| Jasmonate-inducible protein | ml1416.dat | SEQ ID NO: 145 |
| ABA-responsive protein | ml424.dat | SEQ ID NO: 146 |
| Membrane protein | ml843.dat | SEQ ID NO: 147 |
| Dehydration-responsive protein | ml1094.dat | SEQ ID NO: 148 |
| | ml1283.dat | SEQ ID NO: 149 |
| Seed-imbibition protein | ml130.dat | SEQ ID NO: 150 |
| | ml522.dat | SEQ ID NO: 151 |

A sixth group of nucleic acid molecules of the present invention includes DNA sequences that encode portions of proteins for which a putative function has not been assigned.

### EXAMPLE 2

### Hybridization Protocol

The hybridization protocol set forth in this Example is useful, for example, for identifying nucleic acid molecules that hybridize, under stringent hybridization conditions, to one or more of the nucleic acid molecules (or to their complements) that are set forth in SEQ ID NOS:1-472. The hybridization protocol can be used, for example, to screen a cDNA library on a nitrocellulose filter or nylon membrane, and/or to isolate full-length cDNA molecules of the present invention utilizing partial-length cDNA molecules as probes.

Prehybridization solution should be prepared and filtered through a 0.45-micron disposable cellulose acetate filter. The composition of the prehybridization solution is 6×SSC, 5× Denhardt's reagent, 0.5% SDS, 100 μg/ml denatured, fragmented salmon sperm DNA, 50% formamide (alternatively, the formamide may be omitted). When ³²P-labeled cDNA or RNA is used as a probe, poly(A)+ RNA at a concentration of 1 μg/ml may be included in the prehybridization and hybridization solutions to prevent the probe from binding to T-rich sequences that are found fairly commonly in eukaryotic DNA.

Float the nitrocellulose filter or nylon membrane containing the target DNA on the surface of a tray of 6×SSC until it becomes thoroughly wetted from beneath. Submerge the filter for 2 minutes. Slip the wet filter into a heat-sealable bag. Add 0.2 ml of prehybridization solution for each square centimeter of nitrocellulose filter or nylon membrane.

Squeeze as much air as possible from the bag. Seal the open end of the bag with a heat sealer. Incubate the bag for 1-2 hours submerged at the appropriate temperature (65° C. for aqueous solutions; 42° C. for solutions containing 50% formamide). It is desirable to agitate the bag during prehybridization.

If the radiolabeled probe is double-stranded, denature it by heating for 5 minutes at 100° C. Single-stranded probe need not be denatured. Chill the denatured probe rapidly in ice water. Ideally, probe having a specific activity of 109 cpm/μg, or greater, should be used. Typically, hybridization is carried out for 6-8 hours using 1-2 μg/ml radiolabeled probe.

Working quickly, remove the bag containing the filter from the water bath. Open the bag by cutting off one corner with scissors. Add the denatured probe to the prehybridization solution, and then squeeze as much air as possible from the bag. Reseal the bag with the heat sealer so that as few bubbles as possible are trapped in the bag. To avoid radioactive contamination of the water bath, the resealed bag should be sealed inside a second, noncontaminated bag.

When using nylon membranes, the prehybridization solution should be completely removed from the bag and immediately replaced with hybridization solution. The probe is then added and the bag is resealed. Hybridization solution for nylon membranes includes 6×SSC, 0.5% SDS, 100 μg/ml denatured, fragmented salmon sperm DNA, and optionally 50% formamide if hybridization is to be carried out at 42° C. Incubate the bag submerged in a water bath set at the appropriate temperature for the required period of hybridization (for example, twelve hours). Wearing gloves, remove the bag from the water bath and immediately cut off one corner. Pour out the hybridization solution into a container suitable for disposal, and then cut the bag along the length of three sides. Remove the filter and immediately submerge it in a tray containing several hundred milliliters of 2×SSC and 0.5% SDS at room temperature. The filter should not be allowed to dry out at any stage during the washing procedure.

After 5 minutes, transfer the filter to a fresh tray containing several hundred milliliters of 2×SSC and 0.1% SDS and incubate for 15 minutes at room temperature with occasional gentle agitation. The filter should then be washed under the desired, stringent wash conditions. After washing remove most of the liquid from the filter by placing it on a pad of paper towels. Place the damp filter on a sheet of Saran Wrap. Apply adhesive dot labels marked with radioactive ink to several asymmetric locations on the Saran Wrap. These markers serve to align the autoradiograph with the filter. Cover the labels with Scotch Tape. This prevents contamination of the film holder or intensifying screen with the radioactive ink. Radioactive ink is made by mixing a small amount of ³²P with waterproof black drawing ink. Use a fiber-tip pen to apply ink to the adhesive labels.

Cover the filter with a second sheet of Saran Wrap, and expose the filter to X-ray film (Kodak XAR-2 or equivalent) to obtain an autoradiographic image. The exposure time should be determined empirically.

### EXAMPLE 3

### Screening a cDNA Expression Library

This method is used to transfer many bacterial colonies simultaneously from the surface of an agar plate to a nitrocellulose filter. The method works with bacterial colonies of any size, but small colonies (0.1-0.2 mm) give the best results: They produce sharper signals and smear less than larger colonies. As many as 2×10⁴ colonies per 150-mm plate can be screened by this technique. Colonies containing expression vectors carrying the lac promoter should be grown at 37° C. Colonies containing expression vectors carrying the bacteriophage λ p_{R} promoter should be grown at 30° C. to prevent the expression of fusion proteins.

After the bacterial colonies have grown to a diameter of 0.1-0.2 mm, remove the plate from the incubator and store it for 1-2 hours at 4° C. in an inverted position. Label a dry, sterile nitrocellulose filter (Millipore HAWP or equivalent) with a soft-lead pencil or ballpoint pen and place it, numbered side down, on the surface of the agar medium, in contact with the bacterial colonies, until it is completely wet. Mark the filter in at least three asymmetric locations by stabbing through it and into the agar underneath with an 18-gauge needle attached to a syringe containing waterproof black ink.

To induce the expression of a gene cloned into a plasmid carrying the lac promoter, transfer the filter, numbered side up, to a fresh agar plate containing isopropylthio-β-D-galactoside (IPTG). Incubate the plate for 2-4 hours at 37° C. To induce synthesis in expression vectors that carry the bacteriophage λ p_{R} promoter (e.g., the pEX vectors), transfer the filter to a prewarmed plate and incubate for 2-4 hours at 42° C. Remove the filter, and process it for immunological screening as described below. Incubate the master plate for 6 hours at 37° C. (or 30° C.) to allow the colonies to regenerate. Wrap the plate in Saran Wrap and store it at 4° C. in an inverted position until the results of immunological screening are available.

Using blunt-ended forceps (e.g., Millipore forceps), remove the nitrocellulose filters from the plates and place them on damp paper towels. Cover the filters with a plastic hood. Place in the plastic hood an open glass petri dish containing chloroform. Expose the bacterial colonies on the filters to chloroform vapor for 15 minutes.

Transfer small groups of filters to petri dishes containing lysis buffer (6 ml per 82-mm filter; 12 ml per 138-mm filter). When all of the filters have been submerged, stack the petri dishes on a rotary platform and agitate the lysis buffer by gentle rotation of the platform. Lysis of the bacterial colonies takes 12-16 hours at room temperature. The composition of lysis buffer is as follows: 100 mM Tris.Cl (pH 7.8), 150 mM NaCl, 5 mM MgCl₂, 1.5% bovine serum albumin, 1 μg/ml pancreatic DNAase I, 40 μg/ml lysozyme.

Transfer the filters to petri dishes or glass trays containing TNT. Incubate for 30 minutes at room temperature. The composition of TNT is as follows: 10 mM Tris.Cl (pH 8.0), 150 mM NaCl and 0.05% Tween 20. Repeat using fresh TNT. Transfer the filters, one by one, to a glass tray containing TNT. Use Kimwipes to wipe off the residue of the colonies from the surfaces of the filters. Do not allow the filters to dry during any of the subsequent steps.

When all of the filters have been removed and rinsed, transfer them one at a time to a fresh batch of TNT. When all of the filters have been transferred, agitate the buffer gently for a further 30 minutes at room temperature. If so desired, the filters may be removed from the buffer at this stage, wrapped in Saran Wrap, and stored for up to 24 hours at 4° C. Using blunt-ended forceps, transfer the filters individually to glass trays or petri dishes containing blocking buffer (i.e., 20% fetal bovine serum in TNT, use 7.5 ml for each 82-mm filter; 15 ml for each 138-mm filter). When all of the filters have been submerged, agitate the buffer slowly on a rotary platform for 30 minutes at room temperature.

Using blunt-ended forceps, transfer the filters to fresh glass trays or petri dishes containing the primary antibody diluted in blocking buffer (7.5 ml for each 82-mm filter; 15 ml for each 138-mm filter). Use the highest dilution of antibody that gives acceptable background yet still allows detection of 50-100 pg of denatured antigen. When all of the filters have been submerged, agitate the solutions gently on a rotary platform for 2-4 hours at room temperature. The antibody solution can be stored at 4° C. and reused several times. Sodium azide should be added to a final concentration of 0.05% to inhibit the growth of microorganisms.

Wash the filters for 10 minutes in each of the following buffers in the order given. Transfer the filters individually from one buffer to the next. Use 7.5 ml of each buffer for each 82-mm filter and 15 ml for each 138-mm filter. TNT+0.1% bovine serum albumin, followed by TNT+0.1% bovine serum albumin+0.1% Nonidet P-40, followed by TNT+0.1% bovine serum albumin.

Detect the antigen-antibody complexes with the radiochemical or chromogenic reagent of choice. For example, use approximately 1 μCi of ¹²⁵I-labeled protein A or immunoglobulin per filter. Radiolabeled protein A is available from commercial sources (sp. act. 30 mCi/mg). Radioiodinated second antibody can be prepared by art-recognized techniques, such as those set forth in Chapter 12 of Sambrook et al. (1989) Molecular Cloning: A Laboratory Manual, 2nd ed., Cold Spring Harbor Laboratory, Cold Spring Harbor, N.Y. Dilute radiolabeled ligands in blocking buffer (7.5 ml for each 82-mm filter; 15 ml for each 138-mm filter). Incubate the filters for 1 hour at room temperature, and then wash them several times in TNT before establishing autoradiographs.

### EXAMPLE 4

### Genetic Transformation of Peppermint

The procedure for genetically transforming peppermint (Mentha X piperita L.) is based on the procedure set forth in Niu et al., Plant Cell Reports 17:165-171, 1998, which publication is incorporated herein by reference.

Plant material and explant sources: in vitro shoot cultures of peppermint (Mentha X piperita L. var. Black Mitcham) plants are initiated from rhizome explants of peppermint plants maintained in a greenhouse. Shoots are obtained by stimulating axillary bud development from these explants. Typically, 3 to 6 weeks after initial culture shoots are of sufficient size to be used as leaf explants for regeneration or transformation experiments, or to be recultured for continued shoot proliferation.

Tissue culture and plant regeneration: Rhizome segments (1 cm) should be surface disinfected in a solution of 20% bleach (1.05% sodium hypochlorite) with Tween-20 (1 ml/liter of solution) for 20 min and then washed with sterile deionized water. The segments are placed onto the surface of a medium including the following basal constituents: Murashige and Skoog (MS) (Physiol. Plant 15:473-497, 1962) salts, 100 mg/liter myo-inositol, 0.4 mg/liter thiamine, 7.5 g/liter bacteriological grade agar and 30 g/liter sucrose, and 0.1 mg/liter N benzyladenine (BA). The medium should be adjusted to pH 5.8 prior to autoclave sterilization. Typically, shoots will elongate from the axillary buds in the rhizome after 3-4 weeks of culture. Shoots about 1 cm in height are recultured onto the same medium at 3- to 4-week intervals. Shoots (about 5-8 cm in height), at the end of a culture passage, are the source of leaf explants for genetic transformation.

Leaves (1 cm or less in length), including portions of the petioles, are excised from the proximal 5-cm region of the shoot. The leaves should be excised horizontally and the edges of the basal portion trimmed. These explants are placed onto the surface of shoot regeneration medium that contains the basal constituents and 25% coconut water, plus a cytokinin (pH 5.8). Thidiazuron is preferably utilized as a cytokinin for organogenesis. Explants or, subsequently, calli can be recultured at 2-week intervals. Callus develops about 5 weeks after culture initiation and shoots are visible shortly thereafter.

For shoot elongation and root initiation, isolated shoots (6-7 mm) are cultured onto rooting medium that contains the basal constituents and 0.01 mg/liter α-naphthaleneacetic acid (pH 5.8). Shoots are recultured every 2 weeks. Two culture passages are required for sufficient shoot elongation and two to three additional passages for sufficient root development to permit successful soil transplantation. Plants in soil are moved either to a growth chamber or a greenhouse and humidity should be gradually reduced to facilitate hardening.

Shoot cultures used as explant sources, or shoots in elongation or rooting stages of culture, can be maintained at 26° C. and 16 h photoperiod at 25 μmol m⁻²s⁻¹. Leaf explants on regeneration medium can be maintained in darkness at 26 C.

Agrobacterium transformation and kanamycin selection: Representative A. tumefaciens strains useful for transforming peppermint are LBA 4404 (Hoekema et al., Nature 303:179-180, 1983) and EHA 105 (Hood et al., Transgen. Res. 2:208-218, 1993). A representative binary vector plasmid useful for transforming peppermint is pBISN 1 (Narasimhulu et al., Plant Cell 8:873-886, 1996). This binary vector contains a neomycin phosphotransferase (nptII) marker gene for kanamycin selection. Agrobacterium strains can be grown at 30° C. on AB-sucrose minimal or YEP agar medium with 50 μg/ml of kanamycin and 10 μg/ml of rifampicin.

An overnight culture (5 ml YEP medium with 25 mg/liter kanamycin, 28° C.) is inoculated with a single Agrobacterium colony isolated from a freshly cultured plate. An aliquot of this culture is used to inoculate a new 50-ml culture that is grown at 28° C. for 3-4 hours to an OD₆₀₀ of 1.0. Entire leaves are submerged into Agrobacterium culture solution and basal portions (with petiole segments) are excised. Explants are additionally wounded by dissecting away the remaining margins of the leaf piece. The leaf explants are then incubated in the bacterial solution for 30 minutes, blotted briefly, and placed onto regeneration medium without antibiotics for a 4- to 5-day cocultivation period in darkness at 26° C. After cocultivation, the explants are washed with sterile water and then transferred to regeneration medium containing 2.0 mg/liter (8.4 μM) thidiazuron with 20 mg/liter kanamycin and 200 mg/liter Ticar (SmithKline Beecham Pharmaceuticals, Philadelphia, Pa.) for selection of transformed plant cells and inhibition of bacteria, respectively. Shoot elongation and rooting medium contains 15 mg/liter kanamycin and 100 mg/liter Ticar.

Shoot regeneration of peppermint plants from leaf explants: leaves from the proximal 5 cm of the shoot are most morphogenetically responsive for adventitious shoot formation. Further, explants from the basal portion of the leaf contain cells with greater organogenetic competence than those in the leaf tip. Organogenesis occurs either directly from cells in the explant or from those in primary callus. Temporally, shoot or primary callus formation occurs rather uniformly from regions of the leaf that have been injured as a consequence of dissection during explant preparation.

BA, zeatin, or 2-iP have been determined to be required for adventitious shoot formation from orange mint explants (Van Eck and Kitto 1990, 1992). Of the cytokinins tested, thidiazuron most effectively induces shoot formation from cells in peppermint leaf explants. Further, thidiazuron suppresses adventitious root formation that occurs naturally from cultured explants.

### EXAMPLE 5

### Physical Mapping of a Plant Genome

The nucleic acid molecules of the present invention can be used to construct a physical map of a plant genome, such as the peppermint plant genome, utilizing the following, representative techniques which are based on techniques disclosed in Plant Genomes: Methods for Genetic Mapping and Physical Mapping, J. S. Beckmann and T. C. Osborn, eds., Kluwer Academic Publishers (1992), which publication is incorporated herein by reference.

Isolation of Genomic DNA

The procedure given here (based on the method of Hamilton et al. (1972) allows simple rapid procedures for isolation of tobacco leaf nuclei. Anal Biochem. 49:48-57), has been used for the isolation of DNA from Arabidopsis, but is generally applicable to other plants. The procedure describes the extraction of DNA from nuclei which is used to eliminate, or at least reduce, the presence of undesirable plastid DNA.

First, harvest 100 g of tissue which has been destarched by placing the plants in the dark for 48 hours. All subsequent steps are performed at 4° C. unless indicated differently. Wash the tissue with ice-cold water and cut into small pieces using a single-edge razor blade. Cover the tissue with ice-cold diethyl ether and stir for 3 minutes, then decant the ether and rinse well with ice-cold water to remove the residual ether. Add 300 ml of buffer A (1 M sucrose, 10 mM Tris-HCl pH 7.2, 5 mM MgCl₂, 5 mM β-mercaptoethanol and 400 μg/ml ethidium bromide). The inclusion of ethidium bromide is essential for the isolation of high-molecular-weight DNA. Homogenize tissue with either a polytron or Waring blender at medium speed for 1-3 minutes.

Filter the homogenate through 4 layers of cheese cloth, then through 2 layers of Miracloth (Calbiochem). Centrifuge the filtrate at 9000 rpm in a Beckman JA-10 or equivalent rotor for 15 minutes. Decant and discard the supernatant and resuspend the pellet in 50 ml of buffer A plus 0.5% Triton X-100 using a homogenizer with a teflon pestle. Transfer to two, 30 ml Corex tubes and centrifuge at 8000 rpm for 10 minutes in a Beckman JS-13 rotor. Repeat the centrifugation step, except centrifugation is at 6000 rpm for 10 minutes. Resuspend the pellet in 10 ml of buffer A plus 0.5% Triton X-100. Layer the crude nuclei over two discontinuous Percoll gradients prepared as follows: 5 ml steps containing 60% (v/v) and 35% (v/v) Percoll A: buffer A. Percoll A is made as follows: 34.23 g sucrose, 1.0 ml, 1 M Tris-HCl (pH 7.2), 0.5 ml of 1 M MgCl, 34 μl of β-mercaptoethanol and Percoll to a final volume of 100 ml.

Once the gradients have been loaded, centrifuge at 2000 rpm in a Beckman JS-13 rotor. After 5 minutes increase speed to 8000 rpm and spin for an additional 15 minutes. The starch will pellet, the nuclei will band at the 35-65% interface and intact chloroplasts will band at the 0-35% interface. Collect the nuclei from the 35-65% interface and dilute with 5-10 volumes of buffer A. Pellet the nuclei by centrifugation at 8000 rpm in a JS-13 rotor for 10 minutes. The nuclei can be visualized by light microscopy following staining with 1% Azure in buffer A (without ethidium bromide).

Resuspend the nuclei in 5 ml of 250 mM sucrose, 10 mM Tris-HCl (pH 8.0), 5 mM MgCl₂, by homogenization. Bring the volume to 20 ml with TE buffer (10 mM Tris-HCl pH 8.0, 1 mM EDTA) and add EDTA (pH 8.0) to a final concentration of 20 mM. Add 1 ml of 20% Sarkosyl (w/v) and Proteinase K to 100 μg/ml. Incubate at 55° C. until the solution clarifies (approximately 2 hours).

Allow the nuclei preparation to cool to room temperature and add 21 g CsCl. When the CsCl has dissolved, add 1 ml of 10 mg/ml ethidium bromide and mix by gentle inversion. Transfer to two quick-seal tubes and centrifuge in a Beckman Ti 70.1 or equivalent rotor at 65,000 rpm at 20° C. for 16-24 hours. Remove the banded DNA with a 15 gauge needle. If the DNA is of high molecular weight the band should be very viscous. Gently extract the ethidium bromide with an equal volume of isopropanol saturated with CsCl. Repeat the extraction until there is no ethidium bromide present in the organic phase. Dialyze the DNA against three changes of 1 liter of TE. Concentrate the DNA by ethanol precipitation.

Construction of Cosmid Libraries

The success in constructing a physical map depends largely on the quality of the libraries employed. Disclosed herein is a protocol for making random shear cosmid libraries. The reason for using mechanical shear is to avoid any potential bias which might be introduced by either the non-random distribution of restriction sites or differential kinetics of cleavage when limit restriction digests are used to prepare the inserts. In practice, neither differential cleavage nor the uneven distribution of restriction sites is likely to be the major factor in contributing to library bias. Nonetheless, even a small fraction of the genome which contains regions with a non-random distribution of restriction sites or sites which are differentially cleaved, will create gaps in the map since these sequences will be selectively lost from the population. The advantage of mechanical shear is that shear forces are not expected to respect local sequence variations and should therefore produce a totally random distribution of fragments.

Preparation of inserts: Bring 50 to 100 μg of nuclear DNA to a total volume of 500 μl with TE (10 mM Tris-HCl pH 8.0, 1 mM EDTA). Shear the DNA to an average size of 50 to 100 kb. Vortexing the DNA for approximately 1 minute at the maximum setting results in a sample with a size average of around 50 to 100 kb (as visualized by ethidium bromide staining following fractionation on a 0.3% agarose gel). The average size can be adjusted by changing both the time and speed of the vortexing step. It may be necessary to optimize the conditions for each DNA preparation. The mean fragment size should be checked by electrophoresis on a 0.3% agarose gel using intact λ DNA as a standard.

Size-fractionate the sheared DNA on a 36 ml 1.25 M to 5.0 M NaCl (w/v NaCl/TE) gradient by centrifugation at 27,000 rpm for 16 hours at 18° C. in a Beckman SW27 or equivalent rotor. Alternatively, either a 10-40% sucrose gradient or agarose gel electrophoresis can be used for size fractionation (Ausubel et al., eds. (1987) Current Protocols in Molecular Biology. New York: Wiley; Maniatis et al. (1982) Molecular Cloning: A Laboratory Manual. Cold Spring Harbor, N.Y.: Cold Spring Harbor Laboratory Press). The sizing step improves the efficiency of the system by minimizing the number of ligation products which are not in the size range for in vitro packaging into bacteriophage λ particles. More importantly, size fractionation reduces the potential for generating cosmids harboring sequences which are non-contiguous in the genome.

Collect 0.5 ml fractions from the gradient. Check the size distribution by running 15 μl of every third fraction on a 0.3% agarose gel. Pool the fractions having a size distribution between 45 and 70 kb and precipitate with an equal volume of isopropanol. For all subsequent steps it is important that the samples be handled gently to avoid further shearing of the fragments. Mixing should done by gentle pipetting. In addition, it is often difficult to resuspend large fragments following ethanol precipitation. It may therefore be necessary to allow the pellets to resuspend overnight at 4° C. Complete drying of the pellets should be avoided since dehydrated pellets are very difficult to resuspend.

Dissolve the pellet in 400 μl of TE (110 mM Tris-HCl pH 8.0, 1 mM EDTA), add 200 μl 7.5 M NH₄OAc (pH 7.5) and precipitate with 800 μl of ethanol. Wash the pellet with 70% ethanol and briefly air-dry.

T4 polymerase repair of sheared DNA: in order to get efficient ligation of sheared DNA it is necessary to produce blunt ends. There are two steps to this procedure, dephosphorylation with calf intestinal phosphatase (CIP), followed by T4 polymerase “polishing” of the ends. The dephosphorylation serves two functions: (i) by removing the 5′ phosphates the likelihood of getting unwanted ligation products due to multiple inserts is greatly reduced; and (ii) the removal of the 3′ terminal phosphates is necessary to get efficient polishing of the ends. This is important since 3′ phosphates are inhibitory to T4 polymerase.

Bring 5 μg of DNA to 40 μl with TE and add the following: 5 μl of 10×HIN buffer (100 mM Tris-HCl pH 7.5, 600 mM NaCl, 66 mM MgCl, 10 mM DTT), 5 μl of 1 M Tris-HCl (pH 9.0) and 2 μl (20 units) of CIP. Incubate for 40 minutes at 37 C. To terminate the reaction add: 130 μl TE, 20 μl 10×STE (100 mM Tris-HCl pH 8.0, 1 M NaCl, 10 mM EDTA), 10 μl 10% SDS. Incubate at 65° C. for 15 minutes. Extract three times with an equal volume of phenol/chloroform (i.e., phenol/chloroform/isoamyl alcohol in the ratio 25:24:1). Precipitate with 0.5 volumes of 7.5 M NH₄OAc (pH 7.5) and 2 volumes of ethanol. Wash the pellet with 70% ethanol, air-dry for 5 minutes and dissolve in 40 μl of TE.

Add the following: DNA in 40 μl of TE, 5 μl of 10×dNTPs (250 μM solution of all four dNTPs), 5 μl of 10×T4 pol buffer (330 mM Tris-OAc pH 7.9, 660 mM KOAc, 100 mM Mg(OAc)₂, 5 mM DTT, 10 mg/ml BSA), 1 μl T4 polymerase (2 units) and incubate at 37° C. for 30 min. Extract twice with phenol/chloroform, ethanol-precipitate the aqueous phase, wash the pellet with 70% ethanol and resuspend in 20 μl of TE.

Blunt end ligation: This protocol is based on the observation that the rate of blunt-end ligation can be increased by over three orders of magnitude in the presence of large polymers such as polyethylene glycol (PEG). Ligations are carried out in the presence of 15% PEG in a total volume of 60 μl. Since PEG-mediated stimulation of the ligation rate occurs over a fairly narrow concentration range (Pheiffer and Zimmerman, “Polymer-stimulated ligation: enhanced blunt- or cohesive-end ligation of DNA or deoxyribooligonucleotides by T4 DNA ligase in polymer solutions” Nucleic Acids Res. 11:7853-7871, 1983), a rather large reaction volume is used to minimize errors associated with pipetting viscous PEG solutions. It should be noted that DNA tends to be readily sedimentable in 15% PEG so centrifugation should be avoided.

Vector DNA is prepared by the method described by Ish-Horowicz and Burke (“Rapid and efficient cosmid cloning” Nucleic Acids Res. 9:2989-2998, 1981). Vector ‘“arms” are prepared by taking two aliquots of the vector, one of which is cleaved with an enzyme which cuts to the right of the cos site and the other with an enzyme with cleaves to the left of the cos site. The vector arms are then dephosphorylated and cut with an enzyme which generates the blunt-end cloning site. The right and left arms are then purified by agarose gel electrophoresis and eluted from the gel slices by the Gene-Clean procedure (Bio 101). While this method of preparing vector requires more enzymatic steps the efficiency is improved since the dephosphorylation prevents the ligation of tandem vectors and therefore suppresses background due to colonies harboring cosmids with no inserts.

To 5 μg of insert DNA in 20 μl of TE add the following: 1 μg of each vector arm, 3 μl of 10× ligase buffer (660 mM Tris-HCl pH 7.5, 50 mM MgCl₂, 50 mM DTT, 10 mM ATP), H₂₀ to 30 μl. Add 1 μl of T4 ligase (5 units) and mix by gentle pipetting. Add 30 μl of 30% PEG 8000 in H₂O and gently mix. Add 1-2 μl of T4 ligase (5-10 units), mix well by gentle pipetting and incubate at 20° C. for 12 to 24 hours. Add 1 μl of 1 μg/μl acrylamide in H₂O (carrier) and precipitate with 13 μl of 5 M NH₄OAc (pH 7.5) and 200 μl of ethanol. Carefully wash the pellet twice with 70% ethanol, air-dry for 5 minutes and resuspend overnight in 10 μl of TE at 4° C.

In vitro packaging of cosmids: several procedures are available for preparing extracts for the in vitro packaging and subsequent introduction of recombinants into host cells (Ausubel et al. eds. (1987) Current Protocols in Molecular Biology, New York: Wiley; Hohn, “DNA as a substrate for packaging into bacteriophage lambda, in vitro” J. Mol. Biol. 98:93-106, 1975; Hohn “in vitro packaging of Σ and cosmid DNA” Meth Enzymol 68:299-309, 1979; Maniatis et al., Molecular Cloning: A Laboratory Manual. Cold Spring Harbor, N.Y.: Cold Spring Harbor Laboratory Press (1982)). Efficiencies in the range of 10⁷-10⁸ recombinants/μg can be reproducibly attained. However many of the strains commonly used for preparing packaging extracts are based on E. coli K 12 and contain the hsd restriction enzyme. In addition, extracts are usually prepared from cells containing mcrA and mcrB restriction activities, which have the potential to bias the packaging of clones having a high degree of methylation. Bias introduced during packaging can be minimized by preparing extracts from restriction-deficient hosts (mcrA⁻, mcrB⁻, hsdR⁻). Alternatively, extracts are commercially available (Stratagene, La Jolla, Calif.) which are mcrA, mcrB, mrr and hsd restriction-deficient. The commercial extracts also provide high packaging efficiencies (10⁹ pfu/μg) and are available in a form which preferentially package recombinants which are 47 to 51 kb in length and therefore maximize the mean insert size.

Package up to 4 μl of the ligation reaction directly using the chosen protocol. Store the library in 500 μl of SM (100 mM NaCl, 10 mM MgCl₂, 50 mM Tris-HCl pH 7.5, 0.01% (w/v) gelatin) at 4° C. over 20 μl of chloroform. Grow an overnight culture of the bacterial cells in liquid LB medium containing 10 mM MgCl₂ and 0.2% (w/v) maltose at 37° C. A representative bacterial strain is DK 1 (Kurnit “Escherichia coli recA deletion strains that are highly competent for transformation and for in vivo phage packaging” Gene 82:313-315, 1989). Subculture the overnight culture into LB plus Mg²⁺ and maltose by diluting 1 ml into 50 ml and incubate at 37° C. Grow to an A₆₀₀ Of 1.0, harvest the cells by centrifugation for 5 minutes at 4,000 g and resuspend the pellet in 10 ml of 10 mM MgCl₂.

Dilute 5 μl of the library into 100 μl of SM. Add 0.2 ml of the host cells, mix gently and incubate at 37° C. for 20 min. Add 1 ml of LB and incubate at 37° C. for 40 min on a roller drum. Plate varying amounts onto LB plates containing the appropriate antibiotic.

Cosmid DNA miniprep procedure: the miniprep procedure disclosed herein is based on the alkali lysis method of Birnboim et al. (Birnboim and Doly, “A rapid alkaline extraction procedure for screening recombinant plasmid DNA” Nucleic Acids Res. 7:1513-1523 (1979)). Most of the modifications are intended to simplify the handling of large numbers of samples. This procedure is based on the use of repetitive dispensers and centrifuges which hold racks of microcentrifuge tubes (Eppendorf model 5414 or Beckman model 12). By using labeled tube holders it is unnecessary to label sets of individual tubes and the number of manipulations is minimized since the samples are handled in groups of ten. The use of repetitive dispensers greatly simplifies the addition of reagents. While this protocol is more time-consuming than procedures where samples are prepared in microtiter plates, it has the advantage that it gives reasonably good yields of relatively pure DNA which can be subsequently used for other purposes such as making probes.

Inoculate 3 ml of LB medium, containing the appropriate antibiotic, with a single colony. The colonies should be freshly plated. Grow the cultures at 37° C. for 18-22 hours on a roller drum. Remove 2.0 ml of the culture into a 2.2 ml Eppendorf tube. Cultures can be poured directly into the tube. Pellet the cells by centrifugation for approximately 1 minute in a microcentrifuge at 12,000 g. Remove the supernatant by aspiration with a drawn-out pipette. Resuspend the pellet (vortex for 15 seconds) in 250 μl of: 50 mM glucose, 10 mM EDTA, 25 mM Tris-HCl (pH 8.0) and incubate on ice for 5 minutes.

Add 250 μl of 0.2 M NaOH, 1% SDS (fresh) and mix by approximately 15 inversions (do not vortex). Cool on ice for 5 minutes. Add 200 μl of 3.0 M NaOAc, pH 4.8 (ice-cold) and mix by approximately 15 inversions (do not vortex). Let sit on ice for 30-60 minutes then pellet the debris by centrifugation for 5-15 minutes. Remove 600 μl of the supernatant into a 1.5 ml Eppendorf tube. Fill the tube with 100% ethanol and mix well. Pellet the DNA by centrifugation for 2-5 minutes, then decant the ethanol by inverting the rack on a tissue. Briefly air-dry the pellet for 5-10 minutes then resuspend in 250 μl of TE(5) (the 5 denotes that the TE contains 5 mM EDTA rather than the usual 1 mM. TE(5) is 10 mM Tris-HCl pH 8.0, 5 mM EDTA). Leave at room temperature for 15 minutes then vortex briefly. Add 250 μl 4.4 M LiCl, mix, and incubate on ice for 30 minutes. Centrifuge for 5 minutes to pellet debris, then remove 450 μl of the supernatant to a new tube. Fill the tube with ethanol, mix by inversion and place at −20° C. for 20 minutes. Spin for 2-5 minutes to pellet the DNA then decant the ethanol. Wash the pellet with 95% ethanol by adding 1 ml of ethanol, centrifuge for 1 to 2 minutes then decant and discard the supernatant. Briefly air-dry the pellet and resuspend overnight at 4° C. in 50 μl of TE. Solubilize the pellet by vortexing briefly. Yield should be 1-3 μg of cosmid DNA. Although the LiCl precipitation is not essential, it is effective for removing residual protein, cell debris, contaminating E. coli DNA and a significant fraction of the RNA. The quality of the DNA is therefore improved, giving a cleaner and more reproducible fingerprint.

Fingerprinting

Fingerprint reactions with Hind IIII and Sau3A: in the protocol described herein, the clones are digested with Hind III and the resultant ends are simultaneously labeled with reverse transcriptase and the appropriate nucleoside triphosphates. Following thermal inactivation, the samples are then cleaved with a second enzyme, Sau3A. The protocol may be modified for any enzyme or combination of enzymes.

There are several considerations for choosing an enzyme(s): the enzyme(s) should be chosen such that the average number of labeled bands is optimal for the statistical detection of overlaps (Lander and Waterman, “Genomic mapping by fingerprinting random clones: a mathematical analysis” Genomics 2:231-239, 1988). When the inserts are prepared by partial digestion with a restriction enzyme it may be desirable to maintain the same cleavage specificity in the fingerprinting reaction to avoid anomalous bands arising from the insert/vector junction. In practice, this is not important when the fingerprint is composed of a large number of bands. The enzymes used should be active in a single buffer to minimize the number of manipulations required. Preferably, restriction enzymes should be used which retain activity during extended incubation and which are readily available at high concentration. The former minimizes problems associated with analyzing gels containing partial digestion products, while the use of concentrated enzymes eliminates potential glycerol effects (i.e., inhibition of activity and star activity). It may be further advisable to avoid restriction enzymes which are know to cleave their recognition sequences at significantly different rates (Gingeras and Brooks, “Cloned restriction/modification system from Pseudomonus aeruginosa” Proc Natl Acad. Sci USA 80:402-406, 1983; Nath and Azzolina (1981) in: Chirikjian J. G. (ed.), Gene Amplification and Analysis, Vol 1, pp. 113-128. NY: Elsevier-North Holland, New York; Thomas and David, “Studies on the cleavage of bacteriophage lambda DNA with EcoRI restriction endonuclease” J. Mol. Biol. 91:315-328, 1975). Differences in the order of 50-fold have been observed for several enzymes. Differential kinetics of cleavage can contribute to differential labeling and to partial digests, both of which can complicate data analysis. On the other hand, if the differential labeling of sites is reproducible, differences in band intensity can be exploited when assigning overlaps.

The following procedure for fingerprinting clones utilizes an enzyme cocktail having the following composition (enough cocktail for 48 clones): 10 μl ³²P-dATP (3000 Ci/mmol), 80 μl water, 20 μl 10×HIN buffer (100 mM Tris-HCL, pH 7.5, 600 mM NaCl, 66 mM MgCl₂, 10 mM DTT), 2 μl RNase (10 mg/ml RNase IA in 10 mM Tris-HCl, pH 7.6, 15 mM NaCl, boiled for 15 minutes) and 10 μl 1 mM ddGTP.

Pre-cool the enzyme cocktail on ice, then add 2 μl Hind III (50-80 units) and 2 μl M-MLV reverse transcriptase (400 units). Add 2 μl of enzyme cocktail into the wells of a pre-cooled microtiter dish (Nuclon 72×10 μl wells) using a Hamilton PB600-1 repetitive dispenser fitted with a disposable tip. Add 0.5 to 1 μl (25-50 ng) of the cosmid mini-prep DNA to each well. Seal the microtiter dish with a glass plate which has been covered with parafilm to ensure a tight seal. Incubate at 37° C. for 45 minutes. Heat-kill the reaction for 30 minutes at 68° C. Following the heat inactivation, cool the microtiter dish on ice.

Add 4 μl of Sau3A cocktail to each well using a Hamilton PB600-1 repetitive dispenser (Sau3A cocktail includes: 200 μl water, 20 μl 10×HIN buffer (100 mM Tris-HCl pH 7.5, 600 mM NaCl, 66 mM MgCl₂, 10 mM DTT) and 50-100 units of Sau3A. Volume should be less than 8 μl to avoid glycerol effects). Re-seal the dish and incubate at 37° C. for 2-3 hours. Stop the reaction by addition of 5 μl of formamide dye to each well (formamide plus 10 mM EDTA and tracking dyes). To an empty well add 1 μl of labeled Sau3A markers (see below) to 10 μl formamide-dye mix. Place the microtiter dish (which should be left uncovered) at 90° C. for 8 minutes.

³⁵S-labelled Sau3A markers are prepared as follows. Mix the following: 20 μl water, 5 μl 10×HIN buffer, 15 μl ³⁵S-dATP (500 Ci/mmol), 6 μl Sau3A-digested λ DNA (0.5 μg/μl), 2 μl 10 mM dGTP, 2.5 μl 10 mM ddTTP and 1 μM-MLV reverse transcriptase (200 units). Incubate at 37° C. for 30 minutes. Add EDTA to 10 mM and store at −20° C.

Fingerprinting gels: since the gels are run with ³⁵S-labeled markers, it is necessary to fix and dry the gels prior to autoradiography. Preferably the gel is dried directly onto the glass plate. Alternatively, the gels may be fixed, transferred to 3 MM paper and dried on a gel dryer. However, binding the gel directly to the glass plate has the advantage that it prevents distortion of the sample wells. Wells can be formed with combs with 60 usable slots which are 4 mm wide and separated by 1 mm. The 1 mm separation between wells is close to the minimal distance which still gives reproducible polymerization. To ensure that the wells form properly the combs are de-gassed and then flooded with N₂ gas, since the level of oxygen present in the pores of the comb is often sufficient to inhibit polymerization of the narrow slots.

Pre-treatment of gel plates: siliconize the larger of the two plates with Sigma coat (dichlorodimethylsilane), by spreading the concentrated solution onto the plate. Let the solution air-dry for approximately 5 minutes, then remove the excess with 70% ethanol. The second plate is treated with methacryloxypropyltrimethoxysilane, which covalently binds the gel to the glass plate. The binding silane is prepared by adding 5 μl of methacryloxypropyltrimethoxysilane to 3 ml of ethanol plus 50 μl of 10% acetic acid. The binding silane is spread directly on the glass plate with a tissue, air-dried for 5-10 minutes and the excess is removed by washing extensively with ethanol.

Gels are prepared as follows. Gels are 4% acrylamide, Tris/borate/EDTA, 8 M urea. To make one gel mix the following: 48 g urea, 10 ml 40% acrylamide (19:1 acrylamide/bisacrylamide), 10 ml 10×TBE (500 mM Tris-borate, pH 8.3, 10 mM EDTA), 44 ml H₂O. Filter the gel mix to remove any insoluble material. To each 100 ml of gel mix add 200 μl TEMED and 200 μl of 10% ammonium (w/v) persulfate. Pour the gel and allow to polymerize for at least 1 hour prior to running.

Load 1 μl of sample per well and 0.5 μl Sau3A markers every seventh well. Run at 45 mA (approximately 1600 V) until the bromophenol blue dye is approximately 2.5 cm from the bottom of the gel. Fix the gel for 15 minutes in 1 liter of 10% acetic acid and then rinse for 15 minutes in 2 liters of water. The gels are dried directly onto the glass plate in a drying oven for 15 to 30 minutes at 80° C. Alternatively, the gels may be dried overnight at room temperature. Autoradiograph for one to several days on Kodak XAR5 film. The exposure time should be determined empirically. The gels are removed from the glass plate by soaking in 20% Countoff (NEN) or a solution of 1% NaOH.

Image analysis of fingerprint autoradiograms: software which has been developed to assist in mapping by fingerprint analysis is readily available (Coulson et al. “Toward a physical map of the nematode Caenorhabditis elegans” Proc. Natl. Acad. Sci. USA 83:7821-7825, 1986; Sulston et al. “Software for genome mapping by fingerprinting techniques” Comput. Applic. Biosci. 4:125-132, 1988; Sulston et al. “Image analysis of restriction enzyme fingerprint autoradiograms” Cabios 5:101-106, 1989). Briefly, input data are attained using a scanning densitometer and an image processing package. The procedure for image processing involves a preliminary densitometric pass to locate band-like features, lane tracking, a precise densitometric pass and alignment of the marker bands with the standard. Following alignment of the markers, a normalized grid is calculated by linear interpolation between nearest markers and used to calculate the band positions for each lane. For interactive editing the band positions are displayed as colored lines superimposed on an image of the autoradiogram. A VAX station II/GP4 (Digital) may be used for the display and editing of the data. The bands are displayed over the marker lanes, together with the bands from a single sample. Using the “mouse” the operator can selectively remove unwanted bands before moving to the next sample lane. As individual lanes are edited, the normalized position of the bands are written to a data base. Clone matching and contig assembly are performed as described in Coulson et al. “Toward a physical map of the nematode Caenorhabditis elegans” Proc. Natl. Acad. Sci. USA 83:7821-7825, 1986; and Sulston et al. “Software for genome mapping by fingerprinting techniques” Comput. Applic. Biosci. 4:125-132, 1988.

Library Screening

It is not expected that a complete map can be assembled based solely on random clone analysis. At some point it is necessary to close the gaps by selecting the missing clones. There are several alternatives for selecting the linking clones by hybridization. Two approaches are described herein: the selection of linking clones with riboprobes from the ends of existing contigs, and using YAC clones to probe a representative collection of cosmids.

The construction of YAC libraries involves the ligation of large DNA fragments (50-1000 kb) into vectors containing selectable markers and the functional components of a eukaryotic chromosome (Murry and Szostak, “Construction of artificial chromosomes in yeast” Nature 305:189-193, 1983). The constructs are transformed into S. cerevisiae where they are replicated with the host chromosomes. Successful construction of a YAC library depends to a large extent on the ability to isolate megabase sized DNA molecules for the preparation of inserts.

Isolation of Mb-sized DNA from protoplasts: the DNA isolation procedure described herein is based on the isolation of protoplasts which are subsequently embedded in low-gelling agarose. The samples are handled in gel plugs to minimize breakage due to shear forces. The gel inserts are treated with a combination of detergents and enzymes which remove cell membranes, RNA and proteins leaving essentially naked DNA. A high concentration of EDTA is used to inactivate cellular nucleases and an extensive proteinase K treatment in the presence of detergents is used to remove proteins.

Harvest 50 g of tissue which has been destarched by placing the plants in the dark for 48 hours. Wash with ice-cold water and cut into small pieces using either a single-edge razor blade or scissors. Add 500 ml of protoplast buffer (2% cellulose, 0.25% macerozyme, 0.5 M mannitol, 8 mM CaCl₂) and place on a rotary shaker. Incubate overnight at room temperature with shaking at 120 rpm. Filter the homogenate through a sieve with 180 μm pores, then through a second sieve with 75 μm pores. The appropriate pore size is dictated by the nuclear volume and must be adjusted accordingly.

Harvest the protoplasts by centrifugation at room temperature for 5 minutes at 3000 rpm in a JS-13 or equivalent rotor. Resuspend the pellet in 100 ml of 0.5 M mannitol, 8 mM CaCl₂. Harvest the protoplasts by centrifugation for 5 minutes in a JS-13 rotor. Repeat the resuspension and centrifugation step. Resuspend the pellet in 10 ml of 0.5 M mannitol and incubate at 37° C. for 5 minutes. Add 7 ml of 2% low-melting-point agarose prepared in 0.5 M mannitol which is held at 45° C. Mix thoroughly and allow to solidify.

Cut the agarose block into small pieces and incubate overnight at 45° C. with 2.5 μg/ml proteinase K in 0.5 M EDTA, 20 mM Tris-HCl (pH 8.0), 2% sarcosyl. Wash the agarose pieces extensively with 10 mM EDTA, 20 mM Tris-HCl (pH 8.0) at room temperature and store at 4° C.

Cloning in YAC vectors: to establish the conditions for partial digestion of high-molecular-weight DNA set up a series of tubes containing approximately 1 μg of agarose embedded DNA per tube. Add serial dilutions of the restriction enzyme in the appropriate buffer which has been prepared without Mg²⁺ (the Mg²⁺ is required for cleavage). Allow the enzyme to diffuse into the gel slice by incubating at 37° C. for 3 hours. Add Mg²⁺ to a final concentration of 6 mM and continue the incubation at 37° C. for 1 hour. To terminate the reaction add 0.5 M EDTA (pH 8.0) to a final concentration of 20 mM and incubate at 65° C. for 10 minutes. The samples are analyzed by CHEF gel electrophoresis using yeast chromosomes and λ ladders as the size standards (Chu et al. “Separation of large DNA molecules by contour-clamped homogeneous electric fields” Science 324:1582-1585, 1986). Electrophoresis is through a 1% agarose gel in 0.5×TBE at 13° C. The gel is run for 20 hours at 200 V using a 60 second switch interval. Photograph the gel and determine the amount of enzyme needed to produce the maximum fluorescence in the 0.5 to 1 Mb range.

Following optimization of the digestion conditions, the reaction is scaled up for 20 μg of DNA in a 200 μl agarose plug. Melt the agarose plug by incubating at 65° C. for 5 minutes then hold at 37° C. Add a 100-fold molar excess of the restricted, dephosphorylated pYAC 4 (Burke et al. “Cloning of large segments of exogenous DNA into yeast by means of artificial chromosome vectors” Science 236:806-812, 1987) vector. Add 50 μl of 5× ligase buffer (250 mM Tris-HCl pH 7.4, 50 mM MgCl₂, 50 mM DTT, 5 mM spermidine, 5 mM ATP, 500 μg/ml BSA) and 20 units of T4 ligase. Mix well and incubate overnight at room temperature. Separate the unligated vector DNA and small molecules by electrophoresis on a 1% low-melting-point agarose gel run for 10 hours at 40 V. The large DNA molecules which remain near the origin of the gel are excised and embedded in a second 1% low-melting gel. The ligation products are then size-fractionated by electrophoresis on a field inversion gel (Carle and Olson “An electrophoretic karyotype for yeast” Proc Natl Acad. Sci USA 82:3756-3760, 1985). Electrophoresis is carried out at 200 V for 15 hours at 14° C. using a 3 second forward pulse and a 1 second reverse pulse. Slices of the gel containing DNA fragments greater than 100 kb are excised for subsequent transformation.

Yeast transformation: inoculate 10 ml of YEPD medium (1% yeast extract, 2% bacto-peptone, 2% glucose) from a single colony of AB1380 (Burke et al. “Cloning of large segments of exogenous DNA into yeast by means of artificial chromosome vectors” Science 236:806-812, 1987). Incubate at 30° C. for 18-24 hours. Subculture 1 ml of the overnight culture into 80 ml of YEPD medium and grow to a density of 10⁷ cells/ml. Harvest the cells by centrifugation at 4,000 g for 5 minutes and wash twice with 50 ml of 1 M sorbitol. Resuspend the pellet in 10 ml of SCEM (1 M sorbitol, 0.1 M sodium citrate pH 5.8, 10 mM EDTA, 30 mM β-mercaptoethanol) and add 100 μl of 10 mg/ml of zymolyase. Incubate at 30° C. for 15 minutes with gentle shaking. Test for spheroplasting by adding one drop of the cell suspension to each of 2 tubes containing either 1 ml of 1 M sorbitol or 1% SDS in water. The spheroplasts will lyse in the 1% SDS, while cells containing an intact cell wall will not. Continue incubation until spheroplasting is evident.

Melt the agarose block containing the ligated DNA at 65° C. to 70° C. for 5 minutes. To 100 μl of spheroplasted cells add 5 μg of carrier DNA and 10 μl of the ligated DNA in the melted agarose. Incubate at room temperature for 10 minutes. Add 1 ml of 20% (w/v) PEG, 10 mM CaCl₂, 10 mM Tris-HCl (pH 7.5). Mix gently and incubate at room temperature for an additional 10 minutes. Harvest the cells by centrifugation at 3000 g for 4 minutes at room temperature. Resuspend the pellet in 150 μl of SOS medium (1 M sorbitol, 0.25% (w/v) yeast extract, 0.5% (w/v) peptone, 10 μg/ml of uridine and tryptophan, 20 μg/ml of adenine, histidine and lysine) and incubate at 30° C. for 20 to 40 minutes.

Add 8 ml of top agar which is held at 48° C., mix by vortexing and spread onto a pre-warmed agar plate. Pre-warming the plates to 37° C. facilitates uniform spreading of the top agar. Top agar includes the following: 2% agar (w/v), 1.0 M sorbitol, 0.67% (w/v) nitrogen base without amino acids (Difco), 20 mg/ml tryptophan, 10 mg/ml adenine, 20 mg/ml histidine, 20 mg/ml lysine. Incubate the plates at 30° C. for 3 to 5 days.

Individual colonies are picked onto agar plates of complete medium lacking uracil and tryptophan which has been supplemented with canavanine (Burke et al. “Cloning of large segments of exogenous DNA into yeast by means of artificial chromosome vectors” Science 236:806-812, 1987; Hirmen et al. “Transformation of yeast” Proc Natl Acad. Sci USA 75:1929-1933, 1978). Canavanine resistance selects against ochre suppression due to the sup-4 gene harbored on the pYAC4 stuffer fragment (Burke et al. “Cloning of large segments of exogenous DNA into yeast by means of artificial chromosome vectors” Science 236:806-812, 1987). Complete medium includes the following: 0.67% (w/v) nitrogen base without amino acids (Difco); 1.0 mM adenine, alanine, asparagine, aspartate, cysteine, glutamate, glycine, methionine, proline; 2.0 mM leucine, serine, threonine; 0.75 mM isoleucine, phenylalanine; 0.5 mM tyrosine; 0.2 mM cystine; 0.3 mM histidine; 1.5 mM lysine; 2.5 mM valine. Plates contain 2% agar.

The positive clones are then picked into Micronic tubes containing complete medium without uracil and grown to saturation. Glycerol is added to a final concentration of 15% and the clones are held for long-term storage at −80° C.

Small-Scale Preparation of Yeast Chromosomal DNA:

DNA from recombinant yeast clones is prepared for CHEF gel analysis according to the agarose plug procedure of Burke et al. (Burke et al. “Cloning of large segments of exogenous DNA into yeast by means of artificial chromosome vectors” Science 236:806-812, 1987; and Carle et al. “An electrophoretic karyotype for yeast” Proc Natl Acad. Sci USA 82:3756-3760, 1985). Inoculate cells into 4 ml of complete media (Sherman et al. Methods in yeast genetics Cold Spring Harbor, N.Y.: Cold Spring Harbor Laboratory (1983)) lacking uracil and incubate overnight at 30° C. on a roller drum. Harvest the cells by centrifugation at 4,000 g for 5 minutes. Wash the cells in SCE buffer (1 M sorbitol, 0.1 M sodium citrate pH 7.0, 60 mM EDTA pH 8.0) and resuspend the pellet in 100 μl of SCEM buffer (SCE plus 70 mM β-mercaptoethanol and 2.5 mg/ml zymolyase T20).

Heat the cells to 37° C. for 5 minutes and add 125 μl of 1.2% low-melting-point agarose in SCE which is held at 42° C. Mix by pipetting and pour the mixture into 100 μl polystyrene molds. Incubate the solidified plugs overnight at 37° C. in a 24-well microliter plate containing 2 ml of SCEM buffer. Remove the SCEM and replace with 2 ml of lysis solution (0.45 M EDTA pH 8.0, 10 mM Tris-HCl pH 8.0, 1% sarkosyl, 1 mg/ml proteinase K). Incubate at 37° C. for 12 to 24 hours. To determine the insert size and for subsequent isolation, the YACs are separated from the yeast chromosomes by CHEF gel electrophoresis. The plugs can be stored for several months in 500 mM EDTA at 4° C.

Library Plating:

The protocols which we describe apply to both randomly spread colonies and ordered grids. Random clones are spread at a density of about 5000 clones per 15 cm plate. Up to 1000 clones may be gridded onto a 10 by 8 cm rectangle. Grids can be prepared by either tooth-picking the clones or stamped in a 96-well microliter configuration using a 96-prong replicator.

Spread the required number of colonies, or gridded clones, onto Biotrans nylon membranes which are placed in contact with the appropriate medium, i.e., LB plates supplemented with antibiotic for bacterial colonies and complete plates lacking uracil for yeast colonies. Grow colonies overnight (37° C. for bacterial colonies and 30° C. for yeast colonies). Duplicate filters are prepared as described by Coulson et at (Coulson et al. “Genome linking with yeast artificial chromosomes” Nature 335:184-186, 1988). Bacterial clones are disrupted and denatured by stacking the filters between sheets of 3 MM paper and autoclaving for 3 minutes on the fast exhaust cycle. No additional treatment is necessary.

Nylon filters containing yeast colonies are prepared for hybridization as described by Brownstein et at (Brownstein et al. “Isolation of single-copy human genes from a library of yeast artificial chromosome clones” Science 244:1348-1351, 1989). Cells are converted to spheroplasts and subsequently lysed by sequentially placing the filters onto the following series of reagent saturated 3 MM paper: lyticase solution (2 mg/ml zymolyase, 1.0 M sorbitol, 0.1 M Na citrate pH 5.8, 10 mM EDTA, 30 mM β-mercaptoethanol) overnight at 30° C., then 10% SDS for 5 minutes at room temperature, then 0.5 M NaOH for 10 minutes at room temperature and 2×SSC, 0.2 M Tris-HCl (pH 7.5) twice at room temperature. The filters are air-dried for 2 hours and irradiated with 1.2 mJ of 260 nm UV light (Church and Gilbert “Genomic sequencing” Proc Natl Acad. Sci USA 81:1991-1995, 1984).

Riboprobes:

This procedure is based on the use of cosmid vectors containing either T3, T7 or Sp6 bacteriophage promoters flanking the cloned genomic DNA. Riboprobes are prepared from the ends of existing contigs and used to isolate linking clones. When a large number of joins must be established the RNA probes are prepared from pools of cosmids. By using mixed probes the number of hybridizations is reduced by N, where N is the number of clones used for generating the probes. The pooled clones are most conveniently prepared from the rows of the library matrix. Briefly, the clones from the ends of the contigs and the unattached clones are picked in microtiter dishes and gridded onto nylon filters using a 96-prong replicator. Probes are systematically prepared from rows of clones and hybridized to the ordered grids. Overlaps can be established based on the hybridization data. The mixed RNA probes are also used to probe different libraries and therefore select clones which are underrepresented in the original library.

The archived clones are recovered from the glycerol stocks and used to grow overnight cultures in LB containing the appropriate antibiotic. The individual cultures are pooled and used to prepare DNA using the cosmid mini-prep procedure. RNA probes are prepared according to the manufacturer's conditions using T3, T7 or Sp6 (Stratagene) polymerase and ³²P-UTP. The reactions are terminated by phenol extraction. The filters are hybridized at 65° C. in 7% SDS, 1 mM EDTA and 250 mM sodium phosphate (pH 7.2) for 12 to 24 hours. Pre-hybridization is for 5 minutes in the same buffer minus the labeled probe. Washing and autoradiography is as described below except the wash temperature is 65° C. to 70° C.

Preparation of cosmid probes by random priming: linearize approximately 50 to 100 ng of cosmid DNA by digestion with the appropriate restriction enzyme in a total reaction volume in 32 μl. Denature the digested sample by boiling for 5 minutes and quickly chill on ice. Add the following: 2 μl of 10 mg/ml BSA, 10 μl OLB (having the composition set forth below), 5 μl ³²P-dATP (3000 Ci/mmol) and 2 units of Klenow fragment. Incubate at room temperature for a minimum of 2.5 hours. The reactions may be left overnight. Separate the unincorporated dNTPs on a Sephadex G-50 spin column. Prior to hybridization, denature the probe by boiling for 2 minutes then quick-chill on ice.

OLB is made by mixing solutions A:B:C in the ratio 100:250:150. The composition of Solution A is 1 ml 1.25 M Tris-HCl (pH 8.0), 125 mM MgCl₂, 5 μl of 100 mM dCTP, dGTP, dTTP. The composition of Solution B is 2 M Hepes pH 6.6 (store at 4° C.). The composition of Solution C is random hexadeoxyribonucleotides at a concentration of 90 A₂₆₀ units/ml.

Labeling of probes in microtiter plates: the protocol given is for probing 96 filters with YAC clones which are labeled by random priming. This protocol can easily be adapted for samples of isolated DNA such as cosmids. The labeling reactions are done in 96-well microtiter plates and multiple transfers are done with a 12-channel pipette. The labeled clones are used for cross-probing between the cosmid clones and the YACs.

Isolation and labeling of YAC clones: separate the YACs from the resident yeast chromosomes by CHEF gel electrophoresis using 1% low-gelling agarose. Cut the YAC clones out of the gel and store at 4° C. until needed. Melt the YAC slices for 5 to 10 minutes at 70° C. Add 10 μl of the melted YAC slice to 20 μl of distilled water in Micronic tubes (Flow Labs). Heat to 100° C. for 5 minutes in a shallow water bath and allow to cool to room temperature. The Micronic tube rack should be covered with aluminum foil during this step. Remove 8 μl into a 96-well microliter plate containing 4 μl of labeling cocktail. Multiple transfers are performed using a 12-channel pipette. Labeling cocktail for 96 clones contains: 300 μl OLB, 60 μl 10 mg/ml BSA, 60 μl H₂O, 25 μl ³²P-dATP (3000 Ci/rnmol) and 150 units of Klenow fragment.

Seal the microtiter plate and incubate at 37° C. for several hours then incubate overnight at room temperature. Incubate at 70° C. for 5 minutes in a water bath. To each well add 90 μl of denaturing solution and mix thoroughly by pipetting. Incubate at room temperature for 10 minutes. The composition of denaturing solution is: 3.6 ml 100 mM EDTA (pH 8.0), 1.8 ml 10 mg/ml of denatured salmon sperm DNA, 0.9 ml 4 M NaOH and 4.5 ml deionized H₂O.

Hybridization of filters: The composition of hybridization solution is: 125 mM sodium phosphate (pH 7.2), 250 mM NaCl, 10% (w/v) PEG 6000, 7% SDS, 1% BSA. Pipette the labeling reactions into tubes containing 11 ml of the hybridization solution. Using the correct tubes and the appropriate test tube rack, the transfers can be done using a 12-channel pipette. Mix well by inversion and spread the hybridization solution in the lid of a microtiter plate. Soak the filter (DNA side up) in the solution and then invert. If desired add a second filter. Cover the filters with a polythene sheet which has been cut to fit just inside the lid. Stack the lids in an air-tight box and incubate overnight at 68° C. without shaking. The lids are stacked by placing each alternate lid at an angle.

Washing filters: washing can be done in stainless steel wire baskets which are slightly larger than the filters. By doing so the numeric order of the filters is maintained. Washing is carried out in relatively large volumes with gentle agitation. Wash twice with 20 mM sodium phosphate (pH 7.2), 5% SDS, 1 mM EDTA for approximately 5 minutes per wash. The buffer is pre-heated to 68° C. and washing is done on a rotary shaker at room temperature. Wash six times in 20 mM sodium phosphate (pH 7.2), 1% SDS, 1 mM EDTA for 5 minutes per wash. The wash buffer is pre-heated to 50° C. Wash once in 3 mM Tris-base at room temperature. Order the filters on sheets of damp 3 MM paper and cover with saran wrap. Autoradiograph at −80° C. with an intensifying screen.

The filters can be stripped for re-probing by incubating in 2 mM Tris-HCl (pH 8.3), 2 mM EDTA, 0.2% SDS at 70° C. for 10 minutes with gentle agitation. The filters are stored at 4° C. in the same buffer. If the filters are stored for long periods of time the storage buffer should be replaced with fresh buffer every couple of months. Using this treatment it is possible to re-use the filters for a minimum of 20 probings.

Locating Genes of the Present Invention on the Plant Genome Physical Map: the foregoing procedures enable construction of a physical map of a plant genome (such as the genome of the peppermint plant). The map is made up of numerous, overlapping DNA fragments and includes the location of restriction enzyme cleavage sites. One way to determine the position of genes of the present invention on the map is to use full-length, or partial length, cDNAs of the invention as hybridization probes with which to screen (utilizing, for example, the techniques set forth in the present Example) the individual YACs or cosmids that were used to construct the map. The YAC or cosmid clone(s) that hybridize to the probe can then be digested with one or more restriction enzymes and the digestion products separated on an agarose gel by electrophoresis. The gel can be blotted and probed with radiolabelled cDNA molecules, for example utilizing the hybridization protocol set forth in Example 2 herein. In this way, the location of genes of the present invention (encoding one or more cDNAs of the invention) can be located on the plant genome physical map.

While the preferred embodiment of the invention has been illustrated and described, it will be appreciated that various changes can be made therein without departing from the spirit and scope of the invention.

1. An isolated nucleic acid molecule that hybridizes under stringent conditions to a nucleic acid molecule consisting of a nucleic acid sequence selected from the group of nucleic acid sequences consisting of SEQ ID NO:1 through SEQ ID NO:472, or that hybridizes under stringent conditions to the complement of a nucleic acid molecule consisting of a nucleic acid sequence selected from the group of nucleic acid sequences consisting of SEQ ID NO:1 thru SEQ ID NO:472.

2. An isolated nucleic acid molecule of claim 1 that hybridizes under stringent conditions to a nucleic acid molecule consisting of a nucleic acid sequence selected from the group of nucleic acid sequences consisting of SEQ ID NO:29, SEQ ID NO:17, SEQ ID NO:21, SEQ ID NO:66, SEQ ID NO:60, SEQ ID NO:4, SEQ ID NO:7, SEQ ID NO:8, and SEQ ID NO:9, or that hybridizes under stringent conditions to the complement of a nucleic acid molecule consisting of a nucleic acid sequence selected from the group of nucleic acid sequences consisting of SEQ ID NO:29, SEQ ID NO:17, SEQ ID NO:21, SEQ ID NO:66, SEQ ID NO:60, SEQ ID NO:4, SEQ ID NO:7, SEQ ID NO:8, and SEQ ID NO:9.

3. A replicable vector comprising a nucleic acid molecule that hybridizes under stringent conditions to a nucleic acid molecule consisting of a nucleic acid sequence selected from the group of nucleic acid sequences consisting of SEQ ID NO:29, SEQ ID NO:17, SEQ ID NO:21, SEQ ID NO:66, SEQ ID NO:60, SEQ ID NO:4, SEQ ID NO:7, SEQ ID NO:8, and SEQ ID NO:9, or that hybridizes under stringent conditions to the complement of a nucleic acid molecule consisting of a nucleic acid sequence selected from the group of nucleic acid sequences consisting of SEQ ID NO:29, SEQ ID NO:17, SEQ ID NO:21, SEQ ID NO:66, SEQ ID NO:60, SEQ ID NO:4, SEQ ID NO:7, SEQ ID NO:8, and SEQ ID NO:9.

4. A host cell comprising a vector of claim 3.

5. An isolated nucleic acid molecule of claim 1 that hybridizes under stringent conditions to a nucleic acid molecule consisting of a nucleic acid sequence selected from the group of nucleic acid sequences consisting of SEQ ID NO:10, SEQ ID NO:11, SEQ ID NO:12, SEQ ID NO:13, SEQ ID NO:14, SEQ ID NO:15, and SEQ ID NO:16, or that hybridizes under stringent conditions to the complement of a nucleic acid molecule consisting of a nucleic acid sequence selected from the group of nucleic acid sequences consisting of SEQ ID NO:10, SEQ ID NO:11, SEQ ID NO:12, SEQ ID NO:13, SEQ ID NO:14, SEQ ID NO:15, and SEQ ID NO:16.

6. A replicable vector comprising a nucleic acid molecule that hybridizes under stringent conditions to a nucleic acid molecule consisting of a nucleic acid sequence selected from the group of nucleic acid sequences consisting of SEQ ID NO:10, SEQ ID NO:11, SEQ ID NO:12, SEQ ID NO:13, SEQ ID NO:14, SEQ ID NO:15, and SEQ ID NO:16, or that hybridizes under stringent conditions to the complement of a nucleic acid molecule consisting of a nucleic acid sequence selected from the group of nucleic acid sequences consisting of SEQ ID NO:10, SEQ ID NO:11, SEQ ID NO:12, SEQ ID NO:13, SEQ ID NO:14, SEQ ID NO:15, and SEQ ID NO:16.

7. A host cell comprising a vector of claim 6.

8. An isolated nucleic acid molecule of claim 1 that hybridizes under stringent conditions to a nucleic acid molecule consisting of a nucleic acid sequence selected from the group of nucleic acid sequences consisting of SEQ ID NO:107, SEQ ID NO:111, SEQ ID NO:102, SEQ ID NO:10, SEQ ID NO:86, SEQ ID NO:76, SEQ ID NO:81, SEQ ID NO:80, SEQ ID NO:95, and SEQ ID NO:97, or that hybridizes under stringent conditions to the complement of a nucleic acid molecule consisting of a nucleic acid sequence selected from the group of nucleic acid sequences consisting of SEQ ID NO:107, SEQ ID NO:111, SEQ ID NO:102, SEQ ID NO:110, SEQ ID NO:86, SEQ ID NO:76, SEQ ID NO:81, SEQ ID NO:80, SEQ ID NO:95, and SEQ ID NO:97.

9. A replicable vector comprising a nucleic acid molecule that hybridizes under stringent conditions to a nucleic acid molecule consisting of a nucleic acid sequence selected from the group of nucleic acid sequences consisting of SEQ ID NO:107, SEQ ID NO:11, SEQ ID NO:102, SEQ ID NO:10, SEQ ID NO:86, SEQ ID NO:76, SEQ ID NO:81, SEQ ID NO:80, SEQ ID NO:95, and SEQ ID NO:97, or that hybridizes under stringent conditions to the complement of a nucleic acid molecule consisting of a nucleic acid sequence selected from the group of nucleic acid sequences consisting of SEQ ID NO:107, SEQ ID NO:111, SEQ ID NO:102, SEQ ID NO:110, SEQ ID NO:86, SEQ ID NO:76, SEQ ID NO:81, SEQ ID NO:80, SEQ ID NO:95, and SEQ ID NO:97.

10. A host cell comprising a vector of claim 9.
